SEQUENCE LISTING (2 sequences)

SEQ ID NO: 1 - DNA, 32681 bp, Artificial
  Synthetic
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggcccgggatcggtgatcaccgatccagacatgataagatacattgatgagtttggacaaaccacaactagaatgcagtgaaaaaaatgctttatttgtgaaatttgtgatgctattgctttatttgtaaccattataagctgcaataaacaagttcccggatcgatcctcattcattcatcaccgcctcggcttgtcacatttttcttgtcttgctctatctttctttggtctgcattcacatttgttgtgctgtaggaagctcatctctcctatgtgctggccttggtgaggtttgatccgcataatctgcatggtgatgttggactcctcagtgggcacacactccaggccctcgtcattgcagcagcccccgcatcgcatcaggggcacacaggatggcttgaagatgtactcgatctcatcagggtactcctggaagatgtccaccagggtctcgattggatggcagtagctgcgctgatagacatccatgaacttcaccacttcgtgatgattctgccctcctccttctgccatgggtgcagcctgggaccacttggcatggtggaggtagagcagcaaggcaaggctccaatgcacccaagacagcagaaagttcatggtttcggatcgatccgtatagtgagtcgtattaggtaccggctgcagttggacctgggagtggacacctgtggagagaaaggcaaagtggatgtcattgtcactcaagtgtatggccagatctcaagcctgccacacctcaacgtgaagccaagggggtgggcctatagactctataggcggtacttacgtcactcttggcacggggaatccgcgttccaatgcaccgttcccggccgcggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtaactataacggtcctaaggtagcgagctagtggatctataacttcgtataatgtatgctatacgaagttatggcggctagtccatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 2 - DNA, 32409 bp, Artificial
  Synthetic
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggcccgggatcggtgatcaccgatccagacatgataagatacattgatgagtttggacaaaccacaactagaatgcagtgaaaaaaatgctttatttgtgaaatttgtgatgctattgctttatttgtaaccattataagctgcaataaacaagttcccggatcgatcctcattcattcatcaccgcctcggcttgtcacatttttcttgtcttgctctatctttctttggtctgcattcacatttgttgtgctgtaggaagctcatctctcctatgtgctggccttggtgaggtttgatccgcataatctgcatggtgatgttggactcctcagtgggcacacactccaggccctcgtcattgcagcagcccccgcatcgcatcaggggcacacaggatggcttgaagatgtactcgatctcatcagggtactcctggaagatgtccaccagggtctcgattggatggcagtagctgcgctgatagacatccatgaacttcaccacttcgtgatgattctgccctcctccttctgccatgggtgcagcctgggaccacttggcatggtggaggtagagcagcaaggcaaggctccaatgcacccaagacagcagaaagttcatggtttcggatcgatccgtatagtgagtcgtattaggtaccggctgcagttggacctgggagtggacacctgtggagagaaaggcaaagtggatgtcattgtcactcaagtgtatggccagatctcaagcctgccacacctcaagtgaagccaagggggtgggcctatagactctataggcggtacttacgtcactcttggcacggggaatccgcgttccaatgcaccgttcccggccgcggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

(19) United States
(12) Patent Application Publication
(10) Pub. No.: US 2005/0191278 A1
(43) Pub. Date: Sep. 1, 2005

(54) Coronary artery disease treatment

(75) Inventors: Milton L. Pressler, Saline, MI (US); Donald M. Black, Princeton, NJ (US); Paul A. Fischer, North Potomac, MD (US); Henrik S. Rasmussen, Arnold, MD (US); Margaret Samyn, Gainesville, FL (US); Grant Yonehiro, San Mateo, CA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignees: GenVec, Inc., Gaithersburg, MD (US); Warner-Lambert Company, Morris Plains, NJ (US)

(21) Appl. No.: 11/104,126

(22) Filed: Apr. 12, 2005

Related U.S. Application Data
(63) Continuation of application No. PCT/US03/36187, filed on Nov. 14, 2003.
(60) Provisional application No. 60/427,129, filed on Nov. 18, 2002.
(60) Provisional application No. 60/426,700, filed on Nov. 15, 2002.

(51) Int. Cl. A61K 48/00; A61M 31/00

(52) U.S. Cl. 424/93.2; 604/500

(57) ABSTRACT

The invention provides a method of treating coronary artery disease in a human patient comprising directly injecting into an ischemic cardiac muscle, via multiple injections to different points of the cardiac muscle, a dose of a pharmaceutical composition comprising a pharmaceutically acceptable carrier and a replication-deficient adenoviral vector comprising a nucleic acid sequence encoding an angiogenic peptide operably linked to a promoter, whereby the coronary artery disease is treated.

### CROSS-REFERENCE TO RELATED APPLICATIONS

This patent application is a continuation of copending International Patent Application No. PCT/US03/36187, filed Nov. 14, 2003, which designates the United States, and which claims the benefit of U.S. Provisional Patent Application No. 60/427,129, filed Nov. 18, 2002, and which also claims the benefit of U.S. Provisional Patent Application No. 60/426,700, filed Nov. 15, 2002.

### FIELD OF THE INVENTION

The invention pertains to a method of treating coronary artery disease in a human patient.

### BACKGROUND OF THE INVENTION

Coronary artery disease (CAD) is a major health problem. Indeed, in the United States alone it is estimated that over 12 million people have coronary artery disease (American Heart Association, 2002 Heart and Stroke Statistical Update: Dallas, Tex.: American Heart Association (2001), and Criqui, M. H., Vasc. Med., 6 (3 Suppl.), 3-7 (2001)). In addition to this striking number, the prevalence of occlusive arterial disease is likely to increase in the United States and other countries in view of the increasing number of older people. Treatment of patients suffering from occlusive arterial disease remains a considerable clinical issue despite advances in both surgical and percutaneous revascularization techniques. Many patients cannot benefit from these therapies because of the anatomic extent and distribution of arterial occlusion. In such patients, new therapeutic strategies have been sought to prevent heart attacks. Some of these new therapeutic strategies have concentrated on the use of gene therapy to deliver growth factors to a diseased patient with the intention of stimulating blood vessel growth.

Angiogenesis, the growth of new blood vessels from pre-existing vessels, is a complex process involving disruption of vascular basement membranes, migration and proliferation of endothelial cells, and subsequent blood vessel formation and maturation. Several mediators (e.g., angiogenic stimulators) are known to elicit angiogenic responses, and administration of these mediators promotes revascularization of ischemic cardiac muscles. Vascular endothelial growth factor (VEGF) is one of the most specific of the known angiogenic mediators due to localization of its receptors almost exclusively on endothelial cells. Receptors for VEGF are upregulated under ischemic conditions, and the administration of recombinant VEGF augments development of collateral vessels and improves function in myocardial ischemic tissue.

The use of angiogenic mediators to stimulate the formation of new blood vessels is well established. For example, studies have shown, in principle, that it is possible to induce neovascularization in vivo using adenoviral vectors encoding VEGF in nonischemic retroperitoneal adipose tissue and nonischemic subcutaneous tissue (see, e.g., Magovern et al., Hum. Gene Ther., 8 (2), 215-27 (1997), and Lubiatowski et al., Plast. Reconstr. Surg., 110 (1), 149-59 (2002)). Although data from well performed large-scale trials in pharmacologic angiogenesis with systemically administered recombinant protein in coronary artery disease is now available (see, e.g., Henry, Circulation, 100, A2509 (1999), Simons et al., Circulation, 105, 788-793 (2002), and Lederman et al., American College of Cardiology Scientific Sessions, Orlando, Fla. (2001)), the data from similar sized trials utilizing a localized gene therapy strategy, particularly an intramyocardial injection strategy, that address issues pertaining to clinical efficacy, have not been reported.

Accordingly, there remains a need for an effective treatment of human patients suffering from coronary artery disease. The invention provides such a treatment method and a pharmaceutical composition useful in the treatment method. These and other advantages of the invention, as well as additional inventive features, will be apparent from the description of the invention provided herein.

### BRIEF SUMMARY OF THE INVENTION

The invention provides a method of treating coronary artery disease (CAD) in a human patient. The method comprises directly injecting into an ischemic cardiac muscle, via multiple injections to different points of the cardiac muscle, a dose of a pharmaceutical composition comprising (a) a pharmaceutically acceptable carrier and (b) a replication-deficient adenoviral vector comprising a nucleic acid sequence encoding an angiogenic peptide and operably linked to a promoter, wherein the dose comprises about 1×10⁸ to about 4×10¹¹ particle units (pu) of replication-deficient adenoviral vector, whereby the coronary artery disease is treated. The concentration of the angiogenic peptide as a result of the inventive method is at least about 100 pg angiogenic peptide per 1 mg of total protein at the injection site at least 24 hours post injection. The invention also provides a pharmaceutical composition comprising (a) a pharmaceutically acceptable carrier and (b) a replication-deficient adenoviral vector comprising a nucleic acid sequence encoding an angiogenic peptide operably linked to a promoter, wherein the concentration of the replication-deficient adenoviral vector is about 1×10⁷ to about 5×10⁷ particle units of the replication-deficient adenoviral vector per μl of the pharmaceutical composition.

### BRIEF DESCRIPTION OF THE DRAWINGS

FIG. 1 is a bar graph that depicts the change in time to at least 1 mm ST-segment depression on exercise electrocardiogram (ECG) from baseline at 12 weeks and 26 weeks post-treatment in accordance with the invention.

FIG. 2 is a bar graph that depicts the change in time to onset of Level 2 angina or termination of the exercise tolerance test (ETT) in the absence of Level 2 angina from baseline at 12 weeks and 26 weeks post-treatment in accordance with the invention.

FIG. 3 is a bar graph that depicts the change in time of total exercise duration during the exercise tolerance test (ETT) from baseline at 12 weeks and 26 weeks post-treatment in accordance with the invention.

FIG. 4 is a plot graph that depicts the mean Canadian Cardiovascular Society (CCS) angina classification for each treatment group at baseline and at 6, 12, and 26 weeks post-treatment in accordance with the invention.

### DETAILED DESCRIPTION OF THE INVENTION

The inventive method provides an efficient and safe therapeutic regimen for producing an angiogenic peptide in a human for the prophylactic or therapeutic treatment of coronary artery disease (CAD). The term “coronary artery disease” refers to any abnormal condition of the coronary arteries that interferes with the delivery of an adequate supply of blood to the cardiac (i.e., heart) muscle or any portion thereof. Typically, CAD is caused by the accumulation of plaque on the arterial walls (i.e., atherosclerosis), particularly in the large and medium-sized arteries serving the heart. Angina, which is a symptom of ischemia, can be caused by, for example, a coronary spasm, which is usually idiopathic (i.e., of unknown cause), or the result of drug use, such as cocaine use. The lack of sufficient blood to some of the tissue of the cardiac muscle results in that tissue not having sufficient oxygen. In other words, some of the cardiac tissue is ischemic, which can lead to dysfunction or tissue cell death. The cardiac tissue, therefore, will not function appropriately and, possibly, will not survive. The inventive method results in the stimulation of blood vessel growth, such that the ischemic cardiac muscle or ischemic portion of the cardiac muscle receives an increased amount of oxygen so that it is less ischemic (e.g., a sufficient amount of oxygen to survive and/or more adequately function), thereby resulting in the treatment of CAD.

The inventive method is directed to providing a treatment for CAD, for example, by enhancing the level of perfusion of blood to an ischemic cardiac muscle. The term “ischemic” refers to tissue that has become hypoxic (i.e., lacks sufficient oxygen), typically as a result of obstruction of the arterial blood supply or inadequate blood flow. The method comprises directly injecting to different points of an ischemic cardiac muscle a dose of a pharmaceutical composition comprising (a) a pharmaceutically acceptable carrier and (b) a replication-deficient adenoviral vector comprising a nucleic acid sequence encoding an angiogenic peptide and operably linked to a promoter. The method comprises directly injecting the pharmaceutical composition into ischemic cardiac muscle, for example, through use of a catheter. Optimal administration strategies, the pharmaceutical composition dosage, and the manner in which treatment efficacy is ascertained are described in detail below.

Ischemic Cardiac Muscle

The inventive method involves the delivery of a nucleic acid sequence encoding an angiogenic peptide to ischemic cardiac muscle. Cardiac muscle consists of three layers of muscle tissue: the epicardium (outer layer), the myocardium (middle layer), and the endocardium (inner layer). The cardiac muscle contains cells, such as cardiac myocytes, that can be infected by the replication-deficient adenoviral vector comprising the nucleic acid sequence encoding an angiogenic peptide and that will allow the nucleic acid sequence to be expressed to produce the angiogenic peptide. Preferably, the ischemic cardiac muscle comprises receptors such that the angiogenic peptide can exert its biological activity on the tissue. In addition, while ischemia can develop due to blockage of vasculature feeding the tissue, ischemia can develop from other insults to the tissue. For example, congestive heart failure is often the result of coronary artery disease, but also can be associated with development of subendocardial ischemia in the absence of occlusion. The detection of an ischemic cardiac muscle can be performed using any suitable method known in the art. Diagnostic tests for cardiac ischemia are well known and include resting, exercise, or ambulatory electrocardiograms, scintigraphic studies (radioactive heart scans), echocardiography, coronary angiography, and positron emission tomography (PET).

Administration Techniques

The invention involves the administration of a pharmaceutical composition comprising the replication-deficient adenoviral vector comprising a nucleic acid sequence encoding an angiogenic peptide in a localized manner to the ischemic cardiac muscle. The pharmaceutical composition is administered to any region of the cardiac muscle, preferably to the cardiac tissue that forms one or more of the heart chambers (i.e., the left atrium, the right atrium, the left ventricle, and the right ventricle). When the pharmaceutical composition is administered to the cardiac tissue that forms a heart chamber, the pharmaceutical composition preferably is administered to all or part of the outer (i.e., “free”) wall and/or septum of the heart chamber. Alternatively, or in addition, the pharmaceutical composition is administered to all or part of the septum of the heart, which is the muscular wall that separates the left and right ventricles. The pharmaceutical composition can be administered to other regions of the cardiac muscle within the heart, including the papillary muscles of the left and right ventricles (e.g., the anterior or posterior papillary muscles). In that the cardiac tissue that forms the left ventricle is the portion of the cardiac muscle that is most susceptible to developing ischemia, the pharmaceutical composition is preferably administered to the cardiac tissue forming the left ventricle.

The administration of the pharmaceutical composition is not restricted to the cardiac tissue of only one region of the cardiac muscle. Indeed, the treatment of coronary artery disease may require administration of a dose (or even two or more doses) of the pharmaceutical composition to two or more regions of the cardiac muscle to effectively deliver the pharmaceutical composition to the affected area so as to treat the coronary artery disease in the human patient. For example, the pharmaceutical composition can be administered to the cardiac muscle forming a ventricle and an atrium, both atria, both ventricles, an atrium and the septum, and/or a ventricle and the septum (preferably, the left ventricle and the septum).

While any suitable means of administering the pharmaceutical composition to the cardiac muscle can be used within the context of the invention, preferably localized administration to the cardiac muscle is accomplished by directly injecting the pharmaceutical composition into the cardiac muscle. By the term “injecting,” it is meant that the pharmaceutical composition is forcefully introduced into the cardiac muscle. Any suitable injection device can be used within the context of the invention. For example, the common medical syringe can be used to directly inject the pharmaceutical composition into the cardiac muscle. To access cardiac muscle, the cardiac muscle can be exposed during a surgical procedure (e.g., minimally invasive surgery) to allow for such injection. Minimally invasive delivery devices allow administration of the pharmaceutical composition to the cardiac muscle while avoiding more invasive medical procedures (such as open-heart surgery). Such devices are capable of accessing cardiac muscle not directly accessible through the skin, for example, via small incisions of less than 5 inches. Minimally invasive injection devices can comprise injector tips which are flexible and steerable to allow access via small incisions to the curved outer surface of an organ, e.g., the heart. An alternative means of non-invasive injection comprises the use of a needleless injection device, such as the Biojector 2000 Needle-Free Injection Management System® administration device available from Bioject, Inc. The pharmaceutical composition can be administered to a cardiac muscle using a catheter or a system involving a catheter (e.g., a navigational system for catheter-based intramyocardial injection of the pharmaceutical composition, such as the Biosense® intramyocardial injection device available from Biosense, Inc.). Endoscopy is similar to catheterization while permitting visualization of the cardiac muscle while administering the pharmaceutical composition. To allow for multiple injections with a specific geometry, a marking system can be employed so that the sites of previous injections are well delineated, such as described in U.S. Pat. Nos. 5,997,509 and 6,322,536. Enhanced cardiac muscle and injection visualization is especially warranted when the pharmaceutical composition is administered to the cardiac muscle via multiple applications in a distinct geometrical pattern.

A single dose of the pharmaceutical composition is administered via multiple injections of the pharmaceutical composition to different points of the cardiac muscle. Any suitable number of injections can be utilized to administer the pharmaceutical composition to the cardiac muscle. The multiple injections typically will number from about 2 injections to about 50 applications or more (including all integers between 2 and 50), depending on the size of the cardiac muscle, the location and extent of ischemic tissue in the cardiac muscle, and the severity of the disease. For example, a single dose of the pharmaceutical composition can be administered in 2, 3, 4, 5, 6, 7, 8, 9, 10, or more injections (e.g., 15, 20, 25, 30, 35, 40, or 45 injections). The dose of the pharmaceutical composition preferably is administered by about 15 to about 50 injections (e.g., about 10 to about 50 injections), more preferable about 10 to about 40 injections (e.g., about 20 to about 40 injections), and most preferably about 20 to about 30 injections (e.g., about 20 to about 25, or about 30 injections). Multiple injections provide an advantage over single injections in that they can be manipulated to conform to a specific geometry defined by the location of ischemic tissue in the cardiac muscle. The administration of a single dose of the pharmaceutical composition is better controlled using multiple injections, and the effectiveness with which any given dose is administered can be maximized. In this way, too, the adenoviral vector, and ultimately angiogenic peptide production, can be targeted to the cardiac muscle or a particular region thereof (such as the ischemic cardiac tissue of the cardiac muscle).

The specific geometry of the multiple injections is defined by the location on or in the cardiac muscle, either in two- or three-dimensional space, where each injection of the pharmaceutical composition is administered. The pattern of injections is selected to effect a sufficiently broad distribution of the replication-deficient adenoviral vector, and ultimately the produced angiogenic peptide, to the ischemic tissue in the cardiac muscle and, desirably, at least immediately adjacent cardiac tissue, such as the region of the cardiac muscle containing the ischemic cardiac tissue (e.g., the cardiac tissue forming the left ventricle). The multiple injections preferably are spaced such that the points of injection are separated by up to about 4 cm (e.g., about 0.5-4 cm), more preferably up to about 3 cm (e.g., about 1-3 cm), and most preferably up to about 2 cm (e.g., about 1-2 cm). With respect to the specific geometry of the multiple injections in two-dimensional space, the specific geometry is defined by a plane (i.e., a cross-section of the cardiac muscle) in which the multiple injections lie. The plane defined by the multiple injections can lie at a constant distance from the surface of the cardiac muscle (i.e., substantially parallel to the surface of the cardiac muscle) or, alternatively, the plane can lie at an angle with respect to the surface of the cardiac muscle. Preferably, a single injection will be administered for about every 0.5-15 cm of the plane, more preferably for about every 1-12 cm² of the plane, and most preferably for about every 1.5-7 cm² of the plane. The depth of the plane is preferably about 1-10 mm, more preferably about 2-7 mm, and most preferably about 3-5 mm, irrespective of the route of injection (e.g., intramyocardial injection). When the pharmaceutical composition is administered, for example, to all or part of the free wall (preferably all of the free wall) of the left ventricle of the cardiac muscle, the pharmaceutical composition preferably is injected to a depth that is midway through the width of the free wall of the left ventricle, which is typically about 10 mm in thickness. In this respect, therefore, the pharmaceutical composition preferably is injected to a depth of about 5 mm with respect to the free wall of the left ventricle (e.g., from the external or internal surface). In three-dimensional space, a single injection preferably is administered for up to about 50 cm³ (e.g., about 0.5-50 cm³) of cardiac muscle, more preferably for up to about 35 cm³ (e.g., about 1-35 cm³) of cardiac muscle, and most preferably for up to about 15 cm³ (e.g., about 3-15 cm³) of cardiac muscle. Furthermore, the multiple injections can define any suitable pattern or specific geometry. Therefore, for example, in two-dimensional space, the multiple injections can define a square whereas in three-dimensional space the multiple injections can define a cube. To aid in consistent, patterned application of the pharmaceutical composition, a grid or template overlay will aid in guiding injections, marking devices can record previous injection points, and imaging techniques can be used to visualize the cardiac muscle.

The multiple injections can be administered in any suitable time frame with any suitable time differential between each injection, although the multiple injections preferably are administered within about 30 minutes (e.g., within about 15, 20, or 25 minutes), more preferably within about 10 minutes (e.g., within about 0.5-10 minutes), even more preferably within about 8 minutes (e.g., within about 1-8 minutes), and even more preferably within about 6 minutes (e.g., within about 3-6 minutes), of each other. Most preferably, all of the multiple injections of the single dose of the pharmaceutical composition are administered to the cardiac muscle within the aforesaid time frames. If desired, the multiple injections can be administered in uninterrupted (i.e., relatively quick) succession or simultaneously (for example, through use of a multi-syringe device such as described in U.S. Pat. No. 5,846,225).

When administering the pharmaceutical composition to the cardiac muscle, it is desirable that the administration is such that blood vessel formation occurs in and/or to the ischemic tissue in the cardiac muscle. For example, the pharmaceutical composition is administered such that the replication-deficient adenoviral vector contacts regions reasonably flanking the occluded portion of the vasculature and an area therebetween, thereby circumventing damaged vasculature. The resulting collateral blood vessels function as a bypass to the vascular occlusion, thereby restoring, at least in part, blood flow to the ischemic cardiac muscle or at least the ischemic tissue in the cardiac muscle. It is not believed to be necessary to have the replication-deficient adenoviral vector actually contact the precise sites of the source and the terminus for collateral blood vessel formation. However, within the context of multiple injections of the pharmaceutical composition, it is desirable that the specific geometry of the multiple injections be defined to allow the replication-deficient adenoviral vector to contact or reach a region including the source, the terminus, and the area therebetween for collateral blood vessel formation, preferably to actually contact the precise sites of the source and the terminus for the collateral blood vessel formation, along with the area therebetween to form a bridge from non-ischemic tissue to ischemic tissue in the cardiac muscle and/or a bridge around damaged vasculature.

Additional features pertaining to the administration of the pharmaceutical composition via multiple injections are described in International Patent Applications WO 93/32859 and WO 01/34179, U.S. Published Patent Application 2001/0041679 A1, and U.S. Pat. No. 6,329,348.

Dosage

The dose of the pharmaceutical composition, and particularly the amount of the replication-deficient adenoviral vector comprising the nucleic acid sequence encoding an angiogenic peptide, will depend on a number of factors, including the size of the ischemic cardiac muscle, the location and extent of ischemic tissue in the cardiac muscle, the extent of any side-effects, and the like. The dosage should be such that any negative side effects desirably are minimized or at least are balanced with the desired therapeutic effect. Desirably, a single dose of the pharmaceutical composition comprises at least about 1×10⁵ particles units (which also is referred to as particles or pu) of the adenoviral vector. The dose can be at least about 1×10⁶ particle units (e.g., about 4×10⁶ to about 4×10¹² particle units), at least about 1×10⁷ particle units, at least about 1×10⁸ particle units (e.g., about 4×10⁸ to about 4×10¹¹ particle units), at least about 1×10⁹ particle units, or at least about 1×10¹⁰ particle units (e.g., about 4×10⁹ to about 4×10¹⁰ particle units) of the adenoviral vector. The dose of the pharmaceutical composition can comprise no more than about 1×10¹⁴ particle units, no more than about 1×10¹³ particle units, no more than about 1×10¹² particle units, no more than about 1×10¹¹ particle units, or no more than about 1×10¹⁰ particle units (e.g., no more than about 1×10⁹ particle units) of the adenoviral vector. In other words, a single dose of the pharmaceutical composition can comprise about 1×10⁶ particle units (pu), 4×10⁶ Pu, 1×10⁷ pu, 4×10⁷ pu, 1×10⁸ pu, 4×10⁸ pu, 1×10⁹ pu, 4×10⁹ pu, 1×10¹ pu, 4×10¹⁰ pu, 1×10¹¹ pu, 4×10¹¹ pu, 1×10¹¹ pu, 4×10¹ pu, 1×10¹² pu, or 4×10¹² pu of the adenoviral vector. A single dose of the pharmaceutical composition preferably comprising about 1×10⁸ to about 4×10¹¹ particle units, more preferably about 1×10¹⁰ to about 9×10¹⁰ particle units, and most preferably about 2×10¹⁰ to about 8×10¹⁰ particle units, of the adenoviral vector. A single dose of the pharmaceutical composition optionally comprises about 3×10¹⁰ to about 5×10¹⁰ particle units (e.g., 4×10¹⁰ particle units) of the adenoviral vector.

Each application of a multiple injection protocol for administration of a single dose to a human patient will include the approximate fraction of the total dose of the pharmaceutical composition such that the aggregation of the individual injections equals a single dose as described above. Thus, if there are five injections to administer a dose of the pharmaceutical composition, the amount of replication-deficient adenoviral vector in each injection desirably is one-fifth of a single dose as described above. Preferably, each injection comprises about 1×10⁹ to about 5×10⁹ particle units of the replication-deficient adenoviral vector comprising the nucleic acid sequence encoding an angiogenic peptide. Most preferably, each injection comprises about 1×10⁹ to about 2×10⁹ particle units of the adenoviral vector. In a particularly preferred embodiment of the invention, when a single dose of 4×10¹⁰ pu of the adenoviral vector is administered via 30 injections of the pharmaceutical composition, each injection contains about 1.3×10⁹ pu of the adenoviral vector. Alternatively, when a single dose of 4×10¹⁰ pu of the adenoviral vector is administered via 25 injections of the pharmaceutical composition, each injection contains about 1.6×10⁹ pu of the adenoviral vector. If a single dose of 4×10¹⁰ pu of the adenoviral vector is administered via 20 injections of the pharmaceutical composition, each injection contains about 2×10⁹ pu of the replication-deficient adenoviral vector.

A single dose of the pharmaceutical composition can have any suitable volume. The volume of a dose of the pharmaceutical composition desirably is about 100 μl to about 20 ml, preferably about 250 μl to about 10 ml, more preferably 500 μl (i.e., 0.5 ml) to about 5 ml, and most preferably about 1 ml to about 5 ml (e.g., about 1 ml, about 2 ml, about 3 ml, about 4 ml, or about 5 ml). Ideally, the volume of the pharmaceutical composition is about 2 ml to about 5 ml or even about 2 ml to about 3 ml (e.g., about 2, 2.5, or 3 ml). Each injection of the multiple injections used to administer the dose of the pharmaceutical composition can be of any suitable volume. Typically, each injection has a volume of about 50 μl to about 500 μl of the pharmaceutical composition. Preferably, each injection has a volume of about 50 μl to about 150 μl of the pharmaceutical composition. More preferably, each injection has a volume of about 75 μl to about 125 μl (e.g., about 100 μl) of the pharmaceutical composition. Each injection of the multiple injections used to administer a single dose of the pharmaceutical composition typically will include the approximate fraction of the total volume of the dose such that the aggregation of the individual injections equals the volume of a single dose as described above.

The concentration of the adenoviral vector in each injection of the pharmaceutical composition depends upon the dose of the pharmaceutical composition (particularly the concentration of the adenoviral vector therein) and the volume of each injection administered to the human patient. Each injection preferably comprises about 1×10⁷ particle units to about 5×10⁷ particle units of the replication-deficient adenoviral vector per 1 μl of the pharmaceutical composition. More preferably, each injection comprises about 1×10⁷ particle units to about 2×10⁷ particle units of the replication-deficient adenoviral vector per 1 μl of the pharmaceutical composition.

Thus, for example, when a total dose of 4×10¹⁰ particle units of the replication-deficient adenoviral vector is administered via 30 injections of 100 μl volume per injection, the concentration of the adenoviral vector per injection is about 1.3×10⁷ pu of the replication-deficient adenoviral vector per 1 μl of the pharmaceutical composition. Alternatively, when the same dose is administered via 25 injections of 100 μl volume per injection, the concentration of the adenoviral vector per injection is about 1.6×10⁷ pu of the replication-deficient adenoviral vector per 1 μl of the pharmaceutical composition. Administration of the same dose via 20 injections of 100 μl volume per injection results in an adenoviral vector concentration of about 2×10⁷ pu of the replication-deficient adenoviral vector per 1 μl of the pharmaceutical composition.

Thus, in a single dose of the pharmaceutical composition involving, for example, an E1A/E1B/E3-deficient adenoviral vector (described in further detail below) comprising the nucleic acid sequence encoding human VEGF₁₂₁, about 1×10⁸ to about 4×10¹¹ adenoviral particles (i.e., particle units of the adenoviral vector) are administered to an ischemic cardiac muscle. Under these conditions, a substantial level of VEGF₁₂₁ production is achieved in the ischemic cardiac muscle without producing the negative side effects associated with systemic administration of the VEGF₁₂₁ protein.

The nature of coronary artery disease impedes the complete prevention of vascular blockage. Indeed, as with most chronic diseases, prolonged treatment involving multiple doses of the pharmaceutical composition may be required to re-establish perfusion in ischemic cardiac muscle damaged by progressive blockage of feeding vasculature. Accordingly, the inventive method can comprise delivering multiple doses of pharmaceutical composition over a period of time to reverse, at least in part, the symptoms and/or effects of coronary artery disease and improve the quality of life of the patient.

The inventive method can be performed in combination with other therapeutic methods to achieve a desired biological or therapeutic effect in a patient. For example, the inventive method can be practiced on a human patient in conjunction with (e.g., before, during, or after) conventional surgery to treat the coronary artery disease in the patient (e.g., by way of surgical repair of blood vessels in the patient).

Evaluation of Treatment Efficacy

The effectiveness of the inventive method in treating coronary artery disease can be ascertained using any suitable parameter, such as those parameters currently used in the clinic to track occlusive arterial disease. Appropriate parameters include exercise electrocardiograms (ECGs), exercise tolerance test (ETT), ^{99m}Tc-sestamibi single photon emission computed tomography (SPECT), and quality of life questionnaires. Any of these parameters, alone or in any combination, can be used to evaluate the efficacy of the treatment of coronary artery disease in accordance with the invention. The parameters identified above, as well as other parameters suitable for evaluating the treatment efficacy of the invention, are described in, for example, Braunwald et al., Heart Disease: A Textbook of Cardiovascular Medicine, W.B. Saunders Company, Philadelphia, Pa. (6^{th} ed. 2001), and Gibbons et al., J. Am. Coll. Cardiol., 30 (1), 260-311 (1997).

An electrocardiogram (ECG) detects and records the electrical activity of the heart during contraction. The standard clinical ECG involves recordings from 12 leads, including three bipolar limb leads, six unipolar precordial leads, and three modified unipolar limb leads. In evaluating ECG-based data, the ST-segment is the part of the electrocardiographic tracing immediately following the QRS complex and merging into the T wave. In patients with myocardial ischemia, the ST-segment tracing typically is more flat (i.e., becomes more horizontal than normal) as the severity of the ischemic response worsens. With progressive exercise, the ST-segment deviates from baseline (i.e., is depressed from baseline), and the patient may develop angina. Accordingly, ST-segment depression is of particular interest as a primary, and, optionally, a secondary parameter. ECGs typically will be used in the context of the invention to evaluate therapeutic response with respect to coronary artery disease.

Parameters measured by an exercise tolerance test (ETT) include total exercise duration, time to onset of Level 2 angina or termination of ETT in the absence of Level 2 angina, peak rate pressure product (heart rate×systolic blood pressure), and time to onset of at least 1 mm ST-segment depression or termination of ETT in the absence of at least 1 mm ST-segment depression at 26 weeks after treatment by the inventive method. The angina scale for ETT testing consists of 4 levels of angina. Level 1 angina is designated if the onset of angina is mild, but recognized as the usual “angina-of-effort” pain or discomfort with which the subject is familiar. In Level 2 angina, the subject experiences the same pain as in Level 1; however; the pain is moderately severe, definitely uncomfortable, but still tolerable. Level 3 angina is designated if the subject experiences severe anginal pain at a level that the subject will wish to stop exercising. In Level 4 angina, the subject experiences unbearable chest pain, which is the most severe pain the subject has felt.

^{99m}Tc-sestamibi SPECT is valuable in evaluation of a number of therapeutic assessments. For example, a ^{99m}Tc-sestamibi SPECT can be used to determine a summed stress score, which is a semiquantitative measure of perfusion obtained by summing the severity scores of hypoperfusion of 20 segments obtained by post-stress images. The severity scoring is defined as: 0=normal, 1=mildly reduced or equivocal, 2=moderately reduced, 3=severely reduced, and 4=absent uptake. ^{99m}Tc-sestamibi SPECT also can be used to establish a summed rest score (SRS) or a left ventricular ejection fraction (LVEF) (i.e., the ratio between stroke volume and end-diastolic volume). A left ventricular end diastolic volume measurement, the extent of hypoperfusion, and the percent myocardium with reversible or irreversible defects also can be evaluated using ^{99m}Tc-sestamibi SPECT. A global wall motion score (GWMS), which is the mean of severity scores of wall motion abnormality of the 20 segments on the post-stress and rest images, and a summed reversibility difference score (SDS), which is the sum of the differences in severity scores of hypoperfusion between each of the 20 segments on the post-stress and rest images, also are possible using this technology.

The Canadian Cardiovascular Society (CCS) angina classification is used to assign a particular class of angina to a patient. A patient experiences Class I angina if ordinary physical activity, such as walking and climbing stairs, does not cause angina. In Class I angina patients, angina only occurs with strenuous, rapid, or prolonged exertion at work. Class II angina involves “slight limitation of ordinary activity.” In Class II angina patients, angina occurs with walking or climbing stairs rapidly, walking uphill, walking or stair climbing after meals, or in cold, or in wind, or under emotional stress. Walking more than 2 blocks and climbing more than 1 flight of ordinary stairs at a normal pace and in normal conditions also is characteristic of Class II angina. Class III angina is designated if a patient experiences “marked limitation or ordinary physical activity.” In Class III angina patients, angina occurs with walking 1 or 2 blocks and climbing 1 flight of stairs in normal conditions and at normal pace. Finally, a patient is said to have Class IV angina if he or she experiences an “inability to carry on any physical activity without discomfort and anginal syndrome may be present at rest.”

Quality of life questionnaires have been developed to supplement ETT measures to provide patient-reported assessment of angina. The Seattle Angina Questionnaire (SAQ) is a self-administered questionnaire used to assess patients' views of their angina-related quality of life. The SAQ consists of five scales: physical limitation, anginal stability, anginal frequency, treatment satisfaction, and disease perception/quality of life, each of which are transformed so that reported scores range from 0 to 100. The scores in each scale are reported individually, and higher scores indicate a better health-related quality of life (see, e.g., Spertus et al., JACC, 25 (2), 333-341 (1995)). All of the questionnaires identified herein are particularly appropriate to use as secondary efficacy parameters in determining the changes in a patient's functional status secondary to his or her treatment effects.

A therapeutic effect resulting from the inventive method can be ascertained in any suitable manner and desirably is ascertained by comparing baseline values to follow-up values for any one or more of the above parameters. By “baseline values” is meant the values determined for each parameter performed in the baseline study recorded prior to treatment in accordance with the invention. By “follow-up values” is meant the values determined for the same parameter(s) as in the baseline study recorded at an appropriate time after treatment in accordance with the invention (e.g., 1 week, 6 weeks, 12 weeks, 26 weeks, 36 weeks, 48 weeks, or 52 weeks post-treatment). Typically, multiple follow-up studies are performed, and, thus, multiple follow-up values for the same parameters are ascertained at different time points post-treatment (e.g., two or more of 1 week, 6 weeks, 12 weeks, 26 weeks, 36 weeks, 48 weeks, and 52 weeks post-treatment). Suitable time points can be determined by the clinician.

Desirably, in accordance with the inventive method, the treatment of coronary artery disease in a human patient is evidenced by one or more of the following results: (a) at least a 5% increase, preferably at least a 10% increase (e.g., at least a 15% increase), in time to onset of at least 1 mm ST-segment depression on exercise electrocardiograms (ECG) or termination of exercise tolerance test (ETT) in the absence of at least 1 mm ST-segment depression at 12 weeks post-treatment compared to time to onset of at least 1 mm additional ST-segment depression on ECG before treatment, (b) at least a 20% increase, preferably at least a 25% increase (e.g., at least a 28% increase), in time to onset of at least 1 mm additional ST-segment depression on ECG or termination of ETT in the absence of at least 1 mm additional ST-segment depression at 26 weeks post-treatment compared to time to onset of at least 1 mm ST-segment depression in ECG before treatment, (c) at least a 20 second increase, preferably at least a 30 second increase, and more preferably at least a 45 second (e.g., at least a 1 minute) increase, in time to onset of at least 1 mm additional ST-segment depression on ECG or termination of ETT in the absence of at least 1 mm additional ST-segment depression at 12 weeks post-treatment compared to time to onset of at least 1 mm additional ST-segment depression in ECG before treatment, (d) at least a 20 second increase, preferably at least a 30 second increase, and more preferably at least a 45 second (e.g., at least a 1 minute) increase, in time to onset of at least 1 mm additional ST-segment depression on ECG or termination of ETT in the absence of at least 1 mm additional ST-segment depression at 26 weeks post-treatment compared to time to onset of at least 1 mm additional ST-segment depression in ECG before treatment, (e) at least a 10% increase, preferably at least a 20% increase (e.g., at least a 22% increase), in time to onset of Level 2 angina or termination of ETT in the absence of Level 2 angina at 12 weeks post-treatment compared to time to onset before treatment, (f) at least a 20% increase, preferably at least a 30% increase (e.g., at least a 37% increase), in time to onset of Level 2 angina or termination of ETT in the absence of Level 2 angina at 26 weeks post-treatment compared to time of onset before treatment, (g) at least a 30 second increase, preferably at least a 45 second increase, and more preferably at least a 1 minute (e.g., at least a 1.5 minute) increase, in time to onset of Level 2 angina or termination of ETT in the absence of Level 2 angina at 12 weeks post-treatment compared to time to onset before treatment, (h) at least a 30 second increase, preferably at least a 45 second increase, and more preferably at least a 1 minute (e.g., at least a 1.5 minute) increase, in time to onset of Level 2 angina or termination of ETT in the absence of Level 2 angina at 26 weeks post-treatment compared to time to onset before treatment, (i) at least a 5% increase, preferably of least a 7% increase, in time of total exercise duration during ETT at 12 weeks post-treatment compared to time of total exercise duration during ETT before treatment, and (j) at least a 10% increase, preferably at least a 15% increase (e.g., at least a 16% increase), in time of total exercise duration during ETT at 26 weeks post-treatment compared to time of total exercise duration during ETT before treatment, (k) at least a 20 second increase, preferably at least a 30 second increase, and more preferably at least a 45 second (e.g., at least a 1 minute) increase, in time of total exercise duration during ETT at 12 weeks post-treatment compared to time of total exercise duration during ETT before treatment, and (1) at least a 20 second increase, preferably at least a 30 second increase, and more preferably at least a 45 second (e.g., at least a 1 minute) increase, in time of total exercise duration during ETT at 26 weeks post-treatment compared to time of total exercise duration during ETT before treatment.

The treatment of coronary artery disease in a human patient alternatively, or in addition, is evidenced by an improvement in time to onset of angina during ETT. In this respect, at least a one minute increase, preferably at least a 3 minute increase (e.g., at least a 4 minute increase or at least a 5 minute increase), in time to onset of angina during ETT can evidence a therapeutic benefit of the treatment in accordance with the invention. A therapeutic benefit also can be measured in terms of the Canadian Cardiovascular Society (CCS) Angina Classification. In this respect, the treatment of coronary artery disease in a human patient alternatively, or in addition, is evidenced by a decrease of at least one angina class as assigned by the CSS Angina Classification at 12 weeks post-treatment and/or a decrease of at least two angina classes at 26 weeks post-treatment compared to the angina class before treatment.

With respect to quality of life questionnaires, such as the Seattle Angina Questionnaire (SAQ), the therapeutic benefit of the treatment in accordance with the invention can be evidenced in terms of the angina stability score, the angina frequency score, and/or disease perception score. Thus, treatment of coronary artery disease in a human patient alternatively, or in addition, is evidenced by (a) at least a 50% increase, preferably at least a 60% increase, and more preferably at least a 70% (e.g., at least a 74%) increase, in the SAQ angina stability score reported by the human patient at 6 weeks post-treatment compared to the angina stability score reported by the human patient before treatment, (b) at least a 50% increase, preferably at least an 70% increase, and more preferably at least an 80% (e.g., at least an 87%) increase, in the SAQ angina stability score reported by the human patient at 12 weeks post-treatment compared to the angina stability score reported by the human patient before treatment, (c) at least a 50% increase, preferably at least a 60% increase, and more preferably at least a 70% (e.g., at least a 76%) increase, in the SAQ angina stability score reported by the human patient at 26 weeks post-treatment compared to the angina stability score reported by the human patient before treatment, (d) at least a 10 point increase, preferably at least a 20 point increase, and more preferably at least a 25 point (e.g., at least a 30 point) increase in the SAQ angina stability score reported by the human patient at 6 weeks post-treatment compared to the angina stability score reported by the human patient before treatment, (e) at least a 10 point increase, preferably at least a 20 point increase, and more preferably at least a 25 point (e.g., at least a 30 point) increase in the SAQ angina stability score reported by the human patient at 12 weeks post-treatment compared to the angina stability score reported by the human patient before treatment, and/or (f) at least a 10 point increase, preferably at least a 20 point increase, and more preferably at least a 25 point (e.g., at least a 30 point) increase in the SAQ angina stability score reported by the human patient at 26 weeks post-treatment compared to the angina stability score reported by the human patient before treatment. Alternatively, or in addition, the treatment of coronary artery disease in a human patient is evidenced by (a) at least a 50% increase, preferably at least an 70% increase, and more preferably at least an 80% (e.g., at least an 87%) increase, in the SAQ angina frequency score reported by the human patient at 6 weeks post-treatment compared to the angina frequency score reported by the human patient before treatment, (b) at least a 50% increase, preferably at least a 60% increase, and more preferably at least a 70% (e.g., at least a 72%) increase, in the SAQ angina frequency score reported by the human patient at 12 weeks post-treatment compared to the angina frequency score reported by the human patient before treatment, (c) at least a 50% increase, preferably at least a 60% increase, and more preferably at least a 70% (e.g., at least a 73%) increase, in the SAQ angina frequency score reported by the human patient at 26 weeks post-treatment compared to the angina frequency score reported by the human patient before treatment, (d) at least a 10 point increase, preferably at least a 15 point increase, and more preferably at least a 20 point (e.g., at least a 25 point) increase in the SAQ angina frequency score reported by the human patient at 6 weeks post-treatment compared to the angina frequency score reported by the human patient before treatment, (e) at least a 10 point increase, preferably at least a 15 point increase, and more preferably at least a 20 point (e.g., at least a 25 point) increase in the SAQ angina frequency score reported by the human patient at 12 weeks post-treatment compared to the angina frequency score reported by the human patient before treatment, and/or (f) at least a 10 point increase, preferably at least a 15 point increase, and more preferably at least a 20 point (e.g., at least a 25 point) increase in the SAQ angina frequency score reported by the human patient at 26 weeks post-treatment compared to the angina frequency score reported by the human patient before treatment. Similarly, the treatment of coronary artery disease in a human patient alternatively, or in addition, is evidenced by (a) at least a 25% increase, preferably at least a 30% increase; and more preferably at least a 40% (e.g., at least a 45%) increase, in the SAQ disease perception score reported by the human patient at 6 weeks post-treatment as compared to the disease perception score reported by the human patient before treatment, (b) at least a 50% increase, preferably at least a 60% increase, and more preferably at least a 70% (e.g., at least a 72%) increase, in the SAQ disease perception score reported by the human patient at 12 weeks post-treatment as compared to disease perception score reported by the human patient before treatment, (c) at least a 50% increase, preferably at least an 60% increase, and more preferably at least a 70% (e.g., at least an 80%) increase, in the SAQ disease perception score reported by the human patient at 26 weeks post-treatment as compared to the disease perception score reported by the human patient before treatment, (d) at least a 5 point increase, preferably at least a 10 point increase, and more preferably at least a 15 point (e.g., at least a 17 point) increase in the SAQ disease perception score reported by the human patient at 6 weeks post-treatment compared to the disease perception score reported by the human patient before treatment, (e) at least a 10 point increase, preferably at least a 15 point increase, and more preferably at least a 20 point (e.g., at least a 25 point) increase in the SAQ disease perception score reported by the human patient at 12 weeks post-treatment compared to the disease perception score reported by the human patient before treatment, and/or (f) at least a 10 point increase, preferably at least a 15 point increase, and more preferably at least a 20 point (e.g., at least a 25 point) increase in the SAQ disease perception score reported by the human patient at 26 weeks post-treatment compared to the disease perception score reported by the human patient before treatment.

The treatment of coronary artery disease in a human patient desirably is evidenced by one or more (in any combination) of the foregoing results, although alternative or additional results of the referenced tests and/or other tests can evidence treatment efficacy.

Adenoviral Vectors

Adenovirus from any origin, any subtype, mixture of subtypes, or any chimeric adenovirus can be used as the source of the viral genome for the replication-deficient adenoviral vector. A human adenovirus preferably is used as the source of the viral genome for the replication-deficient adenoviral vector. The adenovirus can be of any subgroup or serotype. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22-30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. Adenoviral serotypes 1 through 51 are available from the American Type Culture Collection (ATCC, Manassas, Va.). Preferably, the adenoviral vector is of subgroup C, especially serotype 2 or even more desirably serotype 5.

By “replication-deficient” is meant that the adenoviral vector comprises an adenoviral genome that lacks at least one replication-essential gene function (i.e., such that the adenoviral vector does not replicate in typical host cells, especially those in the human patient that could be infected by the adenoviral vector in the course of treatment in accordance with the invention). A deficiency in a gene, gene function, or gene or genomic region, as used herein, is defined as a deletion of sufficient genetic material of the viral genome to impair or obliterate the function of the gene whose nucleic acid sequence was deleted in whole or in part. Deletion of an entire gene region often is not required for disruption of a replication-essential gene function. However, for the purpose of providing sufficient space in the adenoviral genome for one or more transgenes, removal of a majority of a gene region may be desirable. Replication-essential gene functions are those gene functions that are required for replication (e.g., propagation) and are encoded by, for example, the adenoviral early regions (e.g., the E1, E2, and E4 regions), late regions (e.g., the L1-L5 regions), genes involved in viral packaging (e.g., the IVa2 gene), and virus-associated RNAs (e.g., VA-RNA-1 and/or VA-RNA-2). More preferably, the replication-deficient adenoviral vector comprises an adenoviral genome deficient in at least one replication-essential gene function of one or more regions of the adenoviral genome. Preferably, the adenoviral vector is deficient in at least one gene function of the E1 region of the adenoviral genome required for viral replication (denoted an E1-deficient adenoviral vector). In addition to such a deficiency in the E1 region, the recombinant adenovirus also can have a mutation in the major late promoter (MLP), as discussed in International Patent Application WO 00/00628. Most preferably, the adenoviral vector is deficient in at least one replication-essential gene function (desirably all replication-essential gene functions) of the E1 region and at least part of the nonessential E3 region (e.g., an Xba I deletion of the E3 region) (denoted an E1/E3-deficient adenoviral vector). With respect to the E1 region, the adenoviral vector can be deficient in part or all of the E1A region and part or all of the E1B region, e.g., in at least one replication-essential gene function of each of the E1A and E1B regions. When the adenoviral vector is deficient in at least one replication-essential gene function in one region of the adenoviral genome (e.g., an E1- or E1/E3-deficient adenoviral vector), the adenoviral vector is referred to as “singly replication-deficient.” A particularly preferred singly replication-deficient adenoviral vector is that described in the Examples herein; however, an alternatively preferred singly replication-deficient adenoviral vector for use in the context of the invention comprises a deletion of the entire E1 region and part of the E3 region of the adenoviral genome (i.e., nucleotides 355 to 3,511 and 28,593 to 30,470). A particularly preferred adenoviral vector for use in the context of the invention is deleted of approximately nucleotides 356 to 3,329 and 28,594 to 30,469 (based on the adenovirus serotype 5 genome). Alternatively, the adenoviral vector genome preferably is deleted of approximately nucleotides 356 to 3,510 and 28,593 to 30,470 (based on the adenovirus serotype 5 genome). The endpoints defining the deleted nucleotide portions can be difficult to preciously determine and typically will not significantly affect the nature of the adenoviral vector, i.e., each of the aforementioned nucleotide numbers can be +/−1, 2, 3, 4, 5, or even 10 or 20 nucleotides. The adenoviral vector can, for instance, comprise the nucleotide sequence of SEQ ID NO:1.

The adenoviral vector can be “multiply replication-deficient,” meaning that the adenoviral vector is deficient in one or more replication-essential gene functions in each of two or more regions of the adenoviral genome. For example, the aforementioned E1-deficient or E1/E3-deficient adenoviral vector can be further deficient in at least one replication-essential gene function of the E4 region (denoted an E1/E4- or E1/E3/E4-deficient adenoviral vector), and/or the E2 region (denoted an E1/E2- or E1/E2/E3-deficient adenoviral vector), preferably the E2A region (denoted an E1/E2A- or E1/E2A/E3-deficient adenoviral vector). When E4-deficient, the adenoviral vector genome can comprise a deletion of, for example, nucleotides 32,826 to 35,561 (based on the adenovirus serotype 5 genome), optionally in addition to deletions in the E1 region (e.g., nucleotides 356 to 3,329 or nucleotides 356 to 3,510) and/or deletions in the E3 region (e.g., nucleotides 28,594 to 30,469 or nucleotides 28,593 to 30,470). The adenoviral vector, when multiply replication-deficient, especially in replication-essential gene functions of the E1 and E4 regions, preferably includes a spacer element to provide viral growth in a complementing cell line similar to that achieved by singly replication-deficient adenoviral vectors, particularly an E1-deficient adenoviral vector. The use of a spacer in an adenoviral vector is described in, e.g., U.S. Pat. No. 5,851,806 and International Patent Application WO 97/21826.

Desirably, the adenoviral vector requires, at most, complementation of replication-essential gene functions of the E1, E2A, and/or E4 regions of the adenoviral genome for replication (i.e., propagation). However, the adenoviral genome can be modified to disrupt one or more replication-essential gene functions as desired by the practitioner, so long as the adenoviral vector remains deficient and can be propagated using, for example, complementing cells and/or exogenous DNA (e.g., helper adenovirus) encoding the disrupted replication-essential gene functions. In this respect, the adenoviral vector can be deficient in replication-essential gene functions of only the early regions of the adenoviral genome, only the late regions of the adenoviral genome, and both the early and late regions of the adenoviral genome. The adenoviral vector also can have essentially the entire adenoviral genome removed, in which case it is preferred that at least either the viral inverted terminal repeats (ITRs) and one or more promoters or the viral ITRs and a packaging signal are left intact (i.e., an adenoviral amplicon). Suitable replication-deficient adenoviral vectors, including multiply replication-deficient adenoviral vectors, are disclosed in U.S. Pat. Nos. 5,837,511, 5,851,806, 5,994,106, and 6,579,522, U.S. Published Patent Applications 2001/0043922 A1 2002/0004040 A1, 2002/0031831 A1, and 2002/0110545 A1, and International Patent Applications WO 95/34671, WO 97/12986, and WO 97/21826. Ideally, the pharmaceutical composition is virtually free of replication-competent adenovirus (RCA) contamination (e.g., the pharmaceutical composition comprises less than about 1% of RCA contamination). Most desirably, the pharmaceutical composition is RCA-free. Adenoviral vector compositions and stocks that are RCA-free are described in U.S. Pat. Nos. 5,944,106 and 6,482,616, U.S. Published Patent Application 2002/0110545 A1, and International Patent Application WO 95/34671. Ideally, the pharmaceutical composition also is free of E1-revertants when the adenoviral vector is E1-deficient in combination with deficiencies in other replication-essential gene functions of another region of the adenoviral genome, as further described in International Patent Application WO 03/040314.

In addition to modification (e.g., deletion, mutation, or replacement) of adenoviral sequences encoding replication-essential gene functions, the adenoviral genome can contain benign or non-lethal modifications, i.e., modifications which do not render the adenovirus replication-deficient, or, desirably, do not adversely affect viral functioning and/or production of viral proteins, even if such modifications are in regions of the adenoviral genome that otherwise contain replication-essential gene functions. Such modifications commonly result from DNA manipulation or serve to facilitate expression vector construction. For example, it can be advantageous to remove or introduce restriction enzyme sites in the adenoviral genome. Such benign mutations often have no detectable adverse effect on viral functioning. For example, the adenoviral vector can comprise a deletion of nucleotides 10,594 and 10,595 (based on the adenoviral serotype 5 genome), which are associated with VA-RNA-1 transcription, but the deletion of which does not prohibit production of VA-RNA-1.

Furthermore, the adenoviral vector's coat protein can be modified so as to decrease the adenoviral vector's ability or inability to be recognized by a neutralizing antibody directed against the wild-type coat protein. Similarly, the coat protein of the adenoviral vector can be manipulated to alter the binding specificity or recognition of the adenoviral vector for a viral receptor on a potential host cell. Such manipulations can include deletion or substitution of regions of the fiber, penton, hexon, pIIIa, pVI, and/or pIX, insertions of various native or non-native ligands into portions of the coat protein, and the like. Manipulation of the coat protein can broaden the range of cells infected by the adenoviral vector or enable targeting of the adenoviral vector to a specific cell type. The ability of an adenoviral vector to recognize a potential host cell can be modulated without genetic manipulation of the coat protein, i.e., through use of a bi-specific molecule. For instance, complexing an adenovirus with a bispecific molecule comprising a penton base- or fiber-binding domain and a domain that selectively binds a particular cell surface binding site enables the targeting of the adenoviral vector to a particular cell type.

Suitable modifications to an adenoviral vector are described in U.S. Pat. Nos. 5,543,328, 5,559,099, 5,712,136, 5,731,190, 5,756,086, 5,770,442, 5,846,782, 5,871,727, 5,885,808, 5,922,315, 5,962,311, 5,965,541, 6,057,155, 6,127,525, 6,153,435, 6,329,190, 6,455,314, and 6,465,253, U.S. Published Applications 2001/0047081 A1, 2002/0099024 A1, and 2002/0151027 A1, and International Patent Applications WO 95/02697, WO 95/16772, WO 95/34671, WO 96/07734, WO 96/22378, WO 96/26281, WO 97/20051, WO 98/07865, WO 98/07877, WO 98/40509, WO 98/54346, WO 00/15823, WO 01/58940, and WO 01/92549. The construction and propagation of adenoviral vectors is well understood in the art. Adenoviral vectors can be constructed, propagated (e.g., using complementing cell lines, such as the 293 cell line, Per.C6 cell line, or 293-ORF6 cell line), and/or purified using the materials and methods set forth, for example, in U.S. Pat. Nos. 5,965,358, 5,994,128, 6,033,908, 6,168,941, 6,329,200, 6,383,795, 6,440,728, 6,447,995, and 6,475,757, U.S. Published Application 2002/0034735 A1, and International Patent Applications WO 98/53087, WO 98/56937, WO 99/15686, WO 99/54441, WO 00/12765, WO 01/77304, and WO 02/29388, as well as the other references identified herein. Moreover, numerous adenoviral vectors are available commercially.

The nucleic acid sequence is desirably present as part of an expression cassette, i.e., a particular nucleotide sequence that possesses functions which facilitate subcloning and recovery of a nucleic acid sequence (e.g., one or more restriction sites) or expression of a nucleic acid sequence (e.g., polyadenylation or splice sites). The nucleic acid sequence is preferably located in the E1 region (e.g., replaces the E1 region in whole or in part) of the adenoviral genome. For example, the E1 region can be replaced by a promoter-variable expression cassette comprising a nucleic acid sequence encoding an angiogenic peptide. The expression cassette is preferably inserted in a 3′-5′ orientation, e.g., oriented such that the direction of transcription of the expression cassette is opposite that of the surrounding adjacent adenoviral genome. In addition to the expression cassette comprising the nucleic acid sequence encoding an angiogenic peptide, the adenoviral vector can comprise other expression cassettes containing nucleic acid sequences encoding other products, which cassettes can replace any of the deleted regions of the adenoviral genome. The insertion of an expression cassette into the adenoviral genome (e.g., into the E1 region of the genome) can be facilitated by known methods, for example, by the introduction of a unique restriction site at a given position of the adenoviral genome. As set forth above, preferably all or part of the E3 region of the adenoviral vector also is deleted.

Preferably, the nucleic acid sequence encoding the angiogenic peptide further comprises a transcription-terminating region such as a polyadenylation sequence located 3′ of angiogenic peptide coding sequence (in the direction of transcription of the coding sequence). Any suitable polyadenylation sequence can be used, including a synthetic optimized sequence, as well as the polyadenylation sequence of BGH (Bovine Growth Hormone), polyoma virus, TK (Thymidine Kinase), EBV (Epstein Barr Virus), Human Sarcoma Virus-40, and the papillomaviruses, including human papillomaviruses and BPV (Bovine Papilloma Virus). A preferred polyadenylation sequence is the SV40 polyadenylation sequence (Simian Virus 40).

Preferably, the nucleic acid sequence encoding the angiogenic peptide is operably linked to (i.e., under the transcriptional control of) one or more promoter and/or enhancer elements, for example, as part of a promoter-variable expression cassette. Techniques for operably linking sequences together are well known in the art. Any suitable promoter or enhancer sequence can be used in the context of the invention. Suitable viral promoters include, for instance, cytomegalovirus (CMV) promoters, such as the CMV immediate-early promoter (described in, for example, U.S. Pat. Nos. 5,168,062 and 5,385,839), promoters derived from human immunodeficiency virus (HIV), such as the HIV long terminal repeat promoter, Rous sarcoma virus (RSV) promoters, such as the RSV long terminal repeat, mouse mammary tumor virus (MMTV) promoters, HSV promoters, such as the Lap2 promoter or the herpes thymidine kinase promoter (Wagner et al., Proc. Natl. Acad. Sci., 78, 144-145 (1981)), promoters derived from SV40 or Epstein Barr virus, an adeno-associated viral promoter, such as the p5 promoter, and the like. Preferably, the promoter is the CMV immediate-early promoter. A preferred CMV enhancer/promoter transcription control sequence comprises the CMV viral enhancer, CAAT box, TATA box, transcription start site, and 5′ splice site sequences. The CMV sequences are followed (in the direction of transcription of the expression cassette) by an artificial untranslated region (UTR) of 144 base pairs and 3′ splice site sequences. The open reading frame of the nucleic acid encoding an angiogenic factor can follow 3′ splice site sequences and an SV40 early polyadenylation signal, which can be positioned 3′ of the open reading frame to terminate transcription.

The adenoviral vector can comprise heterologous nucleic acid sequences other than that encoding the angiogenic peptide. If the additional nucleic acid sequence (i.e., transgene) confers a prophylactic or therapeutic benefit, the nucleic acid sequence can exert its effect at the level of RNA or protein. The additional nucleic acid sequence is preferably a different modulator of angiogenesis, such as described in International Patent Application WO 02/22176. Alternatively, the additional nucleic acid sequence can encode an antisense molecule, a ribozyme, a protein that affects splicing or 3′ processing (e.g., polyadenylation), or a protein that affects the level of expression of another gene within the cell (i.e., where gene expression is broadly considered to include all steps from initiation of transcription through production of a processed protein), such as by mediating an altered rate of mRNA accumulation or transport or an alteration in post-transcriptional regulation. The additional nucleic acid sequence can encode a chimeric protein for combination therapy. The additional nucleic acid sequence also can encode a factor that acts upon a different target than the angiogenic peptide, thereby providing multifactorial ischemic treatment. Alternatively, the additional nucleic acid sequence can encode a factor that enhances the effect of the angiogenic peptide.

Angiogenic Nucleic Acid Sequence

The replication-deficient adenoviral vector comprises a nucleic acid sequence encoding an angiogenic peptide, which is expressed to produce the angiogenic peptide in the human patient, particularly in the cardiac muscle of the human patient treated in accordance with the invention. Preferably, the angiogenic peptide is a human angiogenic peptide. The angiogenic peptide has an angiogenic effect in the human patient. Angiogenesis is a complex biological phenomenon that relies on several controlled angiogenic processes. Any biological process involved in the stimulation of new blood vessels (e.g., basement membrane breakdown, cell proliferation, cell migration, vessel wall maturation, lumen formation, vessel dilatation, production of mediators, branching of vessels, and the like) is an “angiogenic effect” that can be achieved or modulated by an angiogenic peptide. Preferably, the angiogenic peptide modulates angiogenic processes by acting upon a target molecule. Target molecules refer to, for example, receptors (e.g., growth factor receptors), intracellular signaling molecules, genes, gene products, such as mRNA and proteins, and chemical mediators.

In addition, as the angiogenic peptide preferably acts on a target molecule, an angiogenic peptide desirably acts upon a cellular signal transduction pathway. Different angiogenic processes rely on different effector molecules and signal transduction pathways for regulation. For example, growth factors, such as a vascular endothelial growth factor (VEGF), fibroblast growth factor (FGF), and platelet-derived growth factor (PDGF), act through a tyrosine kinase family receptor system to transmit signals to the cell nucleus. Growth factors, growth factor receptors, and their corresponding signal transduction pathways are described in the Handbook of Experimental Pharmacology, Spawn & Roberts, Eds., V95, Springer-Verlag (1990). Transcription factors, such as hypoxia-inducible factor-1alpha (HIF-1α) and PR39, do not act through tyrosine kinase receptors but, instead, act directly on DNA to promote the production of positive regulators of angiogenesis. Heparin Binding Neurotrophic Factor (HBNF) acts by up-regulating the production of urokinase, thereby promoting angiogenesis.

Typically, the angiogenic peptide is capable of stimulating angiogenesis, i.e., a peptide that aids in the formation and/or quality of new blood vessels. By “stimulating angiogenesis” is meant that angiogenesis is either initiated or enhanced. Therefore, for example, when angiogenesis is not occurring, the angiogenesis can be initiated. However, if angiogenesis is already occurring, angiogenesis can be enhanced or heightened. Stimulators of angiogenesis are variously described in U.S. Pat. Nos. 5,194,596, 5,219,739, 5,338,840, 5,532,343, 5,169,764, 5,650,490, 5,643,755, 5,879,672, 5,851,797, 5,843,775, and 5,821,124; International Patent Applications WO 95/24473 and WO 98/44953; European Patent Documents 476 983, 506 477, and 550 296; Japanese Patent Documents 1038100, 2117698, 2279698, and 3178996; J. Folkman et al., Nature, 329, 671 (1987); Fernandez et al., Circulation Research, 87, 207-213 (2000), and Moldovan et al., Circulation Research, 87, 378-384 (2000).

Preferably, the angiogenic peptide is a VEGF (especially a human VEGF), such as VEGF-A, VEGF-B, VEGF-C, or VEGF-D. VEGF-B is produced as two isoforms (i.e., VEGF₁₆₇ and VEGF₁₈₅) that, in addition to VEGF-A, also appear to bind Flt-1/VEGFR-1. It may play a role in the regulation of extracellular matrix degradation, cell adhesion, and migration through modulation of the expression and activity of urokinase type plasminogen activator and plasminogen activator inhibitor 1 (see, e.g., Pepper et al., Proc. Natl. Acad. Sci. USA, 95 (20), 11709-11714 (1998)). VEGF-C was originally cloned as a ligand for VEGFR-3/Flt-4, which is primarily expressed by lymphatic endothelial cells. In its fully processed form, VEGF-C also can bind KDR/VEGFR-2 and stimulate proliferation and migration of endothelial cells in vitro and angiogenesis in in vivo models (see, e.g., Lymboussaki et al., Am. J. Pathol., 153 (2), 395-403 (1998), and Witzenbichler et al., Am. J. Pathol., 153 (2), 381-394 (1998)). The overexpression of VEGF-C causes proliferation and enlargement of only lymphatic vessels, while blood vessels are unaffected. VEGF-D is structurally very similar to VEGF-C. VEGF-D is reported to bind and activate at least two VEGFRs, i.e., VEGFR-3/Flt-4 and KDRNEGFR-2. It was originally cloned as a c-fos inducible mitogen for fibroblasts and is most prominently expressed in the mesenchymal cells of the lung and skin (see, e.g., Achen et al., Proc. Natl. Acad. Sci. USA, 95 (2), 548-553 (1998)). VEGF-C and VEGF-D also have been claimed to induce increases in vascular permeability in vivo in a Miles assay when injected into cutaneous tissue (see, e.g., International Patent Application WO 98/07832 and Witzenbichler et al., supra).

Preferably, the human VEGF is VEGF-A (sometimes referred to as “VEGF-1”). The VEGF-A gene contains 8 exons and 7 introns that, by alternative splicing, can form at least six isoforms of the protein, any of which are suitable for use in the invention. The longest protein isoform is VEGF₂₀₆, whose mRNA contains the entirety of all eight exons encoding a pre-protein of 232 amino acids, which is processed to the mature form of 206 amino acids. Alternative splicing to produce the different isoforms is focused around exons 6, 7, and 8 (see, e.g., Robinson et al., J. Cell Sci., 114, 853-865 (2001)). The VEGF₁₂₁ isoform results from joining the splice donor at the end of exon 5 directly to the splice acceptor in exon 8, thereby completely eliminating exons 6 and 7. The VEGF₁₆₅ isoform results from joining the splice donor at the end of exon 5 directly to the splice acceptor in exon 7, thereby completely eliminating exon 6. Exon 6 is especially complex with three different potential splice donors which can ligate to exon 7, resulting in the VEGF₂₀₆, VEGF₁₈₉, and VEGF₁₈₃ isoforms, or exon 8, resulting in the VEGF₁₄₅ isoform. Most preferably, the VEGF is VEGF₁₂₁. Other isoforms of VEGF will be apparent to those skilled in the art. Accordingly, the human VEGF isoforms identified herein are in no way limiting.

While it is preferred that the nucleic acid sequence encoding the angiogenic peptide is wild-type, i.e., will code for a wild-type protein, many modifications and variations of the nucleic acid sequence are possible and appropriate in the context of the invention. For example, the degeneracy of the genetic code allows for the substitution of nucleotides throughout polypeptide coding regions, as well as in the translational stop signal, without alteration of the encoded polypeptide. Such substitutable sequences can be deduced from the known amino acid sequence of the angiogenic peptide or nucleic acid sequence encoding the angiogenic peptide and can be constructed by conventional synthetic or site-specific mutagenesis procedures. Synthetic DNA methods can be carried out in substantial accordance with the procedures of Itakura et al., Science, 198, 1056-1063 (1977), and Crea et al., Proc. Natl. Acad. Sci. USA, 75, 5765-5769 (1978). Site-specific mutagenesis procedures are described in Sambrook et al., Molecular Cloning, A Laboratory Manual, 2d edition, Cold Spring Harbor Press, Cold Spring Harbor, N.Y. (1989).

In addition, a nucleic acid sequence encoding a homolog of the angiogenic peptide, i.e., any protein that is more than about 70% identical (preferably more than about 80% identical, more preferably more than about 90% identical, and most preferably more than about 95% identical) to the protein at the amino acid level and displays angiogenic activity (desirably the same or greater angiogenic activity as VEGF₁₂₁), can be incorporated into the replication-deficient adenoviral vector. The degree of amino acid identity can be determined using any method known in the art, such as the BLAST sequence database. Furthermore, a homolog of the protein can be any peptide, polypeptide, or portion thereof, which hybridizes to the protein under at least moderate, preferably high, stringency conditions, and retains angiogenic activity. Exemplary moderate stringency conditions include overnight incubation at 37° C. in a solution comprising 20% formamide, 5×SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1×SSC at about 37-50° C., or substantially similar conditions, e.g., the moderately stringent conditions described in Sambrook et al., supra. High stringency conditions are conditions that, for example (1) use low ionic strength and high temperature for washing, such as 0.015 M sodium chloride/0.0015 M sodium citrate/0.1% sodium dodecyl sulfate (SDS) at 50° C., (2) employ a denaturing agent during hybridization, such as formamide, for example, 50% (v/v) formamide with 0.1% bovine serum albumin (BSA)/0.1% Ficoll/0.1% polyvinylpyrrolidone (PVP)/50 mM sodium phosphate buffer at pH 6.5 with 750 mM sodium chloride, 75 mM sodium citrate at 42° C., or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at (i) 42° C. in 0.2×SSC, (ii) 55° C. in 50% formamide, and (iii) 55° C. in 0.1×SSC (preferably in combination with EDTA). Additional details and an explanation of stringency of hybridization reactions are provided in, e.g., Ausubel et al., Current Protocols in Molecular Biology, Green Publishing Associates and John Wiley & Sons, New York, N.Y. (1994).

The nucleic acid sequence can encode a functional portion of the angiogenic peptide, i.e., any portion of the protein that retains the biological activity of the naturally occurring, full-length protein at measurable levels. A functional angiogenic peptide fragment produced by expression of the nucleic acid sequence of the replication-deficient adenoviral vector can be identified using standard molecular biology and cell culture techniques, such as assaying the biological activity of the fragment in human cells transiently transfected with a nucleic acid sequence encoding the protein fragment.

Alternatively, the nucleic acid sequence encoding the angiogenic peptide can be manipulated to alter (preferably enhance) the activity of the angiogenic peptide. The nucleic acid sequence can be manipulated to enhance secretion of the angiogenic peptide, or can be manipulated to encode an angiogenic peptide that remains bound to the cell surface. The nucleic acid sequence can be manipulated to enhance the stability of the angiogenic peptide (e.g., retain proper three-dimensional conformation under adverse conditions), or to increase the potency of the angiogenic peptide for activation of a receptor specific for the angiogenic peptide. As such, the invention is not limited to the wild-type nucleic acid sequence, and modifications of the nucleic acid sequence are contemplated herein.

The replication-deficient adenoviral vector comprising a nucleic acid sequence encoding an angiogenic peptide operably linked to a promoter desirably is administered to the cardiac muscle in a quantity such that the angiogenic peptide is produced at sufficient levels in the cardiac muscle at an appropriate time following the administration of the pharmaceutical composition to the cardiac muscle so as to elicit a therapeutic effect against coronary artery disease. The concentration of the angiogenic peptide achieved as a result of the use of the invention typically is at least about 100 pg angiogenic peptide (e.g., at least about 200 pg, at least about 300 pg, at least about 400 pg, or at least about 500 pg angiogenic peptide) per 1 mg of total protein at the injection site at least 24 hours (e.g., at least 2, at least 3, or at least 4 days) post injection. The concentration of the angiogenic peptide can be about 100 pg to about 1400 pg angiogenic peptide per 1 mg of total protein at the injection site, about 100 pg to about 700 pg angiogenic peptide per 1 mg of total protein at the injection site, or even about 100 pg to about 300 pg angiogenic peptide per 1 mg of total protein at the injection site. Alternatively, the concentration of the angiogenic peptide can be about 800 pg to about 1400 pg angiogenic peptide per 1 mg of total protein at the injection site, about 800 pg to about 1000 pg (i.e., 1 ng) angiogenic peptide per 1 mg of total protein at the injection site, or even about 2 ng to about 4 ng (e.g., about 2 ng to about 4 ng, or about 4 ng to about 6 ng) angiogenic peptide per 1 mg of total protein at the injection site. Such concentrations of angiogenic peptide desirably are maintained in the cardiac muscle for at least 12 hours, e.g., at least 24 hours, at least about 2 days, or at least about 3 days. The concentration of the angiogenic peptide can be determined using any suitable method known in the art for measuring protein levels, such as, for example, Western blot, enzyme-linked immunosorbent assay (ELISA), the BCA assay (Smith et al., Anal. Biochem., 150, 76-85 (1985)), the Lowry protein assay (described in, e.g., Lowry et al., J. Biol. Chem., 193, 265-275 (1951)), which is a calorimetric assay based on protein-copper complexes, and the Bradford protein assay (described in, e.g., Bradford et al., Anal. Biochem., 72, 248 (1976)), which depends upon the change in absorbance in Coomassie Blue G-250 upon protein binding. When the angiogenic peptide is VEGF₁₂₁, VEGF₁₂, levels can be quantified using the VEGF ELISA kit provided by R&D Systems, Inc., Minneapolis, Minn. In the context of the invention, protein levels “at the injection site” typically have reference to the tissue about a 1 cm³ central portion surrounding the injection site. Similarly, the concentration of the angiogenic peptide about 1 cm from the site of injection (for example, in 1 cm³ portions of cardiac muscle at sites adjacent to the injection site (e.g., at sites 1 cm to the left or right of the injection site)), which evidences the distribution or “spread” of the angiogenic protein within the cardiac muscle, typically is about 5 pg to about 150 pg angiogenic peptide per 1 mg of total protein at the injection site.

Pharmaceutical Composition

The inventive method involves the administration of a pharmaceutical composition comprising a pharmaceutically acceptable carrier and a replication-deficient adenoviral vector comprising a nucleic acid sequence encoding an angiogenic peptide in a localized manner to the ischemic cardiac muscle. Any suitable pharmaceutically acceptable carrier can be used within the context of the invention, and such carriers are well known in the art. The choice of carrier will be determined, in part, by the particular site to which the pharmaceutical composition is to be administered and the particular method used to administer the pharmaceutical composition.

Suitable formulations include aqueous and non-aqueous solutions, isotonic sterile solutions, which can contain anti-oxidants, buffers, bacteriostats, and solutes that render the formulation isotonic with the blood or other bodily fluid of the human patient, and aqueous and non-aqueous sterile suspensions that can include suspending agents, solubilizers, thickening agents, stabilizers, and preservatives. Preferably, the pharmaceutically acceptable carrier is a liquid that contains a buffer and a salt. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. When the formulation is presented in a vial, the vial can be constructed of any material suitable for housing a pharmaceutical composition. The vial desirably is constructed of glass or plastic. The viral preferably is a glass vial. Glass vials are typically constructed of clear glass or tinted glass (e.g., green, blue, or amber), so as to protect a light-sensitive pharmaceutical composition contained therein from degradation. Preferably, the pharmaceutically acceptable carrier is a buffered saline solution.

The pharmaceutical composition can be maintained, formulated, packaged, and/or presented within a catheter, rather than, for example, a vial or ampule. Alternatively, the pharmaceutical composition can be maintained within a drug delivery cassette. Ideally, if the pharmaceutical composition is maintained within a drug delivery cassette, the drug delivery cassette can be placed into a catheter, which allows for release and dispersal of the pharmaceutical composition within the catheter.

The pharmaceutical composition preferably is formulated to protect the adenoviral vector from damage prior to administration. The particular formulation desirably decreases the light sensitivity and/or temperature sensitivity of the adenoviral vector. Indeed, the pharmaceutical composition will be maintained for various periods of time and, therefore, should be formulated to ensure stability and maximal activity at the time of administration. The pharmaceutical composition can be maintained as a frozen formulation, that is, at a temperature below 0° C. The pharmaceutical composition desirably is maintained at a temperature above 0° C. and/or as a liquid, preferably at 4° C. or higher (e.g., 4-10° C.). It can be desirable to maintain the pharmaceutical composition at a temperature of 10° C. or higher (e.g., 10-20° C.), 20° C. or higher (e.g., 20-25° C.), or even 30° C. or higher (e.g., 30-40° C.). The pharmaceutical composition can be maintained at the aforementioned temperature(s) for at least 1 day (e.g., 7 days (1 week) or more), though typically the time period will be longer, such as at least 3, 4, 5, or 6 weeks, or even longer, such as at least 10, 11, or 12 weeks, prior to administration to a patient. During that time period, the adenoviral gene transfer vector optimally loses no, or substantially no, activity, although some loss of activity is acceptable, especially with relatively higher storage temperatures and/or relatively longer storage times. The activity of the adenoviral vector composition desirably decreases about 50% or less, preferably about 20% or less, more preferably about 10% or less, and most preferably about 5% or less, after any of the aforementioned time periods.

To this end, the pharmaceutical composition preferably comprises a pharmaceutically acceptable liquid carrier, such as, for example, those described above, and a stabilizing agent selected from the group consisting of polysorbate 80, L-arginine, polyvinylpyrrolidone, α-D-glucopyranosyl α-D-glucopyranoside dihydrate (commonly known as trehalose), and combinations thereof. More preferably, the stabilizing agent is trehalose, or trehalose in combination with polysorbate 80. The stabilizing agent can be present in any suitable concentration in the pharmaceutical composition. When the stabilizing agent is trehalose, the trehalose desirably is present in a concentration of about 1-25% (wt./vol.), preferably about 2-10% (wt./vol.), and more preferably about 4-6% (wt./vol.) of the pharmaceutical composition. When trehalose and polysorbate 80 are present in the pharmaceutical composition, the trehalose preferably is present in a concentration of about 4-6% (wt./vol.), and more preferably about 5% (wt./vol.), while the polysorbate 80 desirably is present in a concentration of about 0.001-0.015% (wt./vol.), preferably about 0.001-0.01% (wt./vol.), and more preferably about 0.0025% (wt./vol.). When a stabilizing agent, e.g., trehalose, is included in the pharmaceutical composition, the pharmaceutically acceptable liquid carrier preferably contains a saccharide other than trehalose. The pharmaceutical composition can further comprise about 0.05-3 mM (e.g., about 0.05-2 mM, about 0.05-1.8 mM, about 0.05-1.5 mM, about 0.5-1.5 mM, or about 0.05-1 mM), preferably about 1 mM or about 2 mM, of a divalent metal salt and/or a cationic polymer to further stabilize the pharmaceutical composition. Desirably, the divalent metal salt is MgCl₂. In addition, the pharmaceutical composition can further comprise a suitable amount of NaCl, such as about 50-150 mM, preferably about 75 mM NaCl. The liquid carrier typically is water, preferably water suitable for infection (WFI). Suitable formulations of the pharmaceutical composition are further described in U.S. Pat. No. 6,225,289, U.S. Pat. No. 6,514,943, U.S. Published Patent Application 2002/0019041 A1, U.S. Published Patent Application 2003/0153065 A1, International Patent Application WO 00/34444, and International Patent Application WO 03/59292. The adenoviral vector most preferably is dispersed in an aqueous formulation buffer containing 5.5% (wt./vol.) trehalose, 0.0025% (wt./vol.) polysorbate 80, 2 mM MgCl₂, 75 mM NaCl, and 10 mM Tris HCl (pH 7.6±0.02).

The pharmaceutical composition can further be formulated to reduce adherence loss of the adenoviral vector on devices used to prepare, store, or administer the expression vector, such as glassware, syringes, or needles. Use of such a pharmaceutical composition can extend the shelf life of the pharmaceutical composition, facilitate administration, and increase the efficacy of the inventive method. In this regard, the pharmaceutical composition also can be formulated to enhance the spread of the adenoviral vector throughout the ischemic cardiac muscle and/or enhance transduction efficiency. To this end, the pharmaceutical composition also can comprise hyaluronidase, which has been shown to enhance uptake of adenoviral vectors. It is desirable to formulate the composition such that the replication-deficient adenoviral vector remains in the ischemic cardiac muscle and does not leak into surrounding normal tissue, i.e., to increase retention of the pharmaceutical composition in the ischemic cardiac muscle. Accordingly, agents that increase viscosity of the pharmaceutical composition, such as stimuli-sensitive polymers, can be included in the pharmaceutical composition. Alternatively, or in addition, the adenoviral vectors of the pharmaceutical composition can be bound to biocompatible solid carriers (e.g., a stent), such as particulate carriers (e.g., beads, wafers, etc.), that remain in the ischemic cardiac muscle due to size, or incorporated into a matrix, such as gel or foam.

In addition, the pharmaceutical composition can comprise additional therapeutic or biologically active agents. For example, therapeutic factors useful in the treatment of a particular indication can be present. Factors that control inflammation, such as ibuprofen or steroids, can be part of the pharmaceutical composition to reduce swelling and inflammation associated with in vivo administration of the adenoviral vector and physiological distress. Immune system suppressors can be administered with the pharmaceutical composition to reduce any immune response to the adenoviral vector itself or associated with a disorder. Alternatively, immune enhancers can be included in the pharmaceutical composition to up regulate the body's natural defenses against disease. Vitamins and minerals, anti-oxidants, and micronutrients can be co-administered with the pharmaceutical composition. Antibiotics, i.e., microbicides and fungicides, can be present to reduce the risk of infection associated with gene transfer procedures and other disorders.

### EXAMPLES

The following examples further illustrate the invention but, of course, should not be construed as in any way limiting its scope. These examples are directed to clinical studies involving the administration of a pharmaceutical composition of the invention in the treatment of coronary artery disease. Each study consisted of three phases: a screening phase, a baseline phase, and a follow-up phase. The pharmaceutical composition used in each instance contained (a) a pharmaceutically acceptable carrier and (b) a replication-deficient adenoviral vector comprising a nucleic acid sequence encoding VEGF₁₂₁. The dosages and administration protocol varied in each case.

The adenoviral vector in each study was a human adenovirus of serotype 5 in which part of the E1 region, part of the VA-1 region, and part of the E3 region of the adenoviral genome (i.e., approximately nucleotides 356 to 3,329, 10,594 to 10,595, and 28,594 to 30,469) were deleted (i.e., an E1A/E1B/E3-deficient human adenoviral serotype 5 viral backbone comprising a deletion in the VA-1 region) and comprising the coding sequence for human VEGF₁₂₁ located in the E1 region and operably linked to the CMV promoter (AdVEGF121). The adenoviral vector was dispersed in an aqueous formulation buffer containing 3% (wt./vol.) sucrose, 10 mM MgCl₂, 150 mM NaCl, and 10 mM Tris (pH 7.8) (Chesapeake Biological Laboratories, Inc., Baltimore, Md.). The pharmaceutical composition was prepared in each case by the Clinical Pharmaceutical Operations at Parke-Davis Pharmaceutical Research. The follow-up studies were performed at about the same time of day (i.e., within about 2 hours) as the corresponding baseline studies.

### EXAMPLE 1

This example demonstrates the safety and efficacy of a pharmaceutical composition of the invention delivered through minimally invasive surgery versus maximum medical treatment in the treatment of patients suffering from advanced coronary artery disease, as measured by comparing time to onset of at least 1 mm ST-segment depression before and after treatment in accordance with the invention.

Patients were screened to allow initial assessment of qualification for randomization into the study (i.e., those with stable and severe angina). Baseline values were established for each qualified patient with respect to exercise electrocardiogram (ECG), (i.e., time to onset of at least 1 mm ST-segment depression), exercise tolerance test (ETT), ^{99m}Tc-sestamibi SPECT (i.e., summed stress score, summed reversibility score, and global wall motion scores), Canadian Cardiovascular Society (CCS) angina class, and the Seattle Angina Questionnaire (SAQ) response.

67 patients (i.e., 32 in Group A and 35 in Group B) with stable and severe angina (CCS Angina Class II-IV, excluding prolonged and unstable angina at rest) were enrolled after screening and baseline procedures at approximately 15 to 25 study centers located in different geographic areas in North America. These patients had been treated with maximum medical therapy for at least 2 months prior to the present study, but the disease failed to be completely controlled, resulting in unacceptable lifestyle limitations. Maximum medical therapy included the following medications (unless hemodynamic parameters or intolerance contraindicated their use): Nitroglycerine, anti-anginal medications (e.g., long-acting nitrates, calcium-channel blockers, and beta-blockers), and platelet aggregation inhibitors (e.g., aspirin, ticlopidine, or clopidogrel). These patients were randomized into 2 groups, namely Group A and Group B, according to a randomization code prepared by the Parke-Davis Biometrics Department. Patients in Group A received 30 epicardial injections of the pharmaceutical composition totaling 4×10¹⁰ pu adenoviral vector (100 μl each injection) at 1.5 to 2 cm intervals covering the left ventricle of the cardiac muscle through minimally invasive surgery. They were hospitalized, monitored daily, and managed according to the current standard of care until recovered from the surgery procedure. Patients in Group B did not receive the pharmaceutical composition or minimally invasive surgery and were not hospitalized.

Assessment of vector levels in the plasma, before and within 15 minutes after the completion of the injection procedure, was performed at selected sites. The assessment of plasma VEGF levels also were performed at Day 3, Week 1 (Day 5-9), Week 2 (Day 12-16), Week 6, (Day 32-46), and Week 26 (Day 170-190) post-treatment at selected sites. Adenoviral cultures were performed at Week 2 post-treatment at selected sites. Follow-up assessments were performed at Week 6, Week 12, and Week 26 post-administration (Group A) and post-randomization (Group B). The study was completed for data analysis when all patients withdrew or completed procedures up to and including the Week 26 visit. All patients were followed up at 1 year (Week 52) for general safety assessment (physical examination, noninvasive cancer screen, and body weight) and incidence of cardiac events (hospitalization for myocardial infarction (MI) or unstable angina).

The initial projected sample size of approximately 35 patients in each treatment group was based on a 2-sided t-test at the 5% level of significance. It was expected to provide 85% power to detect a difference of 2 minutes between treatment groups in terms of the mean change from baseline in the time to onset of at least 1 mm ST-segment depression. The standard deviation of the change from baseline in the time to onset of at least 1 mm additional ST-segment depression was estimated to be 2.5 minutes. A 14% loss due to dropouts was assumed.

Analysis of covariance was performed to compare the effects of the pharmaceutical composition and maximum medical therapy on the change from baseline in the time to onset of at least 1 mm additional ST-segment depression, or termination of ETT in the absence of at least 1 mm additional ST-segment depression, at week 12. The primary model included main effects due to treatment, center, and number of prior coronary artery bypass grafting (CABG) (<2 and ≧2), with baseline value as a covariate. The p-value for the comparison of treatment effects and the 95% confidence interval for the difference in treatment effects were provided. The presence of treatment-by-center, treatment-by-prior CABG, and treatment-by-covariate interactions was investigated. The primary analysis was included from all randomized patients with a baseline assessment and Week 12 assessment. All statistical testing was 2-sided and conducted at the 5% level of significance. The secondary and other parameters were analyzed in the same manner as the primary efficacy parameter.

Summary statistics (mean, standard error, median, minimum, and maximum) for baseline, follow-up, and change from baseline measures were provided for all efficacy parameters. Summary statistics also were computed for demographic parameters for each treatment group.

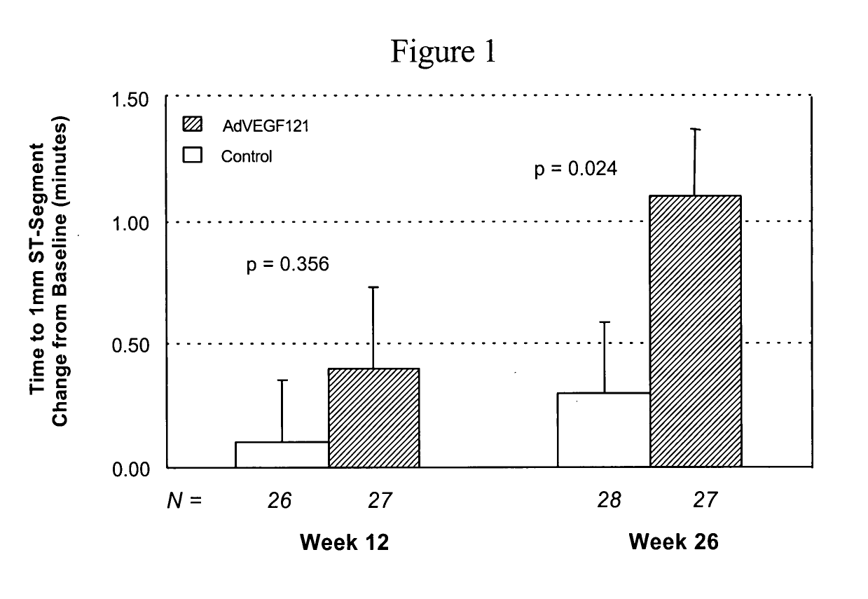

Treatment efficacy was assessed by measuring the change from baseline at Week 12 and/or Week 26 compared to maximum medical therapy in the time to onset of at least 1 mm additional ST-segment depression on exercise ECG, or termination of ETT in the absence of at least 1 mm additional ST-segment depression (“time to 1 mm additional ST-segment depression”). The results of these efficacy measurements are set forth in FIG. 1. The baseline time to 1 mm additional ST segment depression was measured as the mean time to 1 mm additional ST-segment depression in Group A and Group B, which were 3.9 and 4.2 minutes, respectively. Group A included 27 patients and Group B included 29 patients, although evaluable data was not available for all patients at all time points. At 12 weeks post-treatment, an approximate 10% increase in time to onset of at least 1 mm additional ST-segment depression on ECG or termination of ETT in the absence of at least 1 mm additional ST-segment depression was observed in Group A, as compared to time to onset of at least 1 mm additional ST-segment depression on ECG before treatment. At 26 weeks post-treatment, the time to 1 mm additional ST-segment depression increased approximately 28% in Group A, as compared to the time to 1 mm additional ST-segment depression before treatment, which represents a significant change compared to control Group B (p=0.026).

Treatment efficacy also was assessed at Weeks 12 and 26 by measuring the change from baseline in the following: additional ETT parameters (i.e., total exercise duration, time to onset of Level 2 angina or termination of ETT in the absence of Level 2 angina), Canadian Cardiovascular Society (CCS) angina class; and the Seattle Angina Questionnaire (SAQ). The results of the various secondary parameter assessments are set forth in Examples 2-6.

### EXAMPLE 2

This example demonstrates the safety and efficacy of a pharmaceutical composition of the invention delivered through minimally invasive surgery versus maximum medical treatment in the treatment of patients suffering from advanced coronary artery disease, as measured by comparing time to onset of Level 2 angina or termination of the exercise tolerance test (ETT) in the absence of Level 2 angina before and after treatment in accordance with the invention.

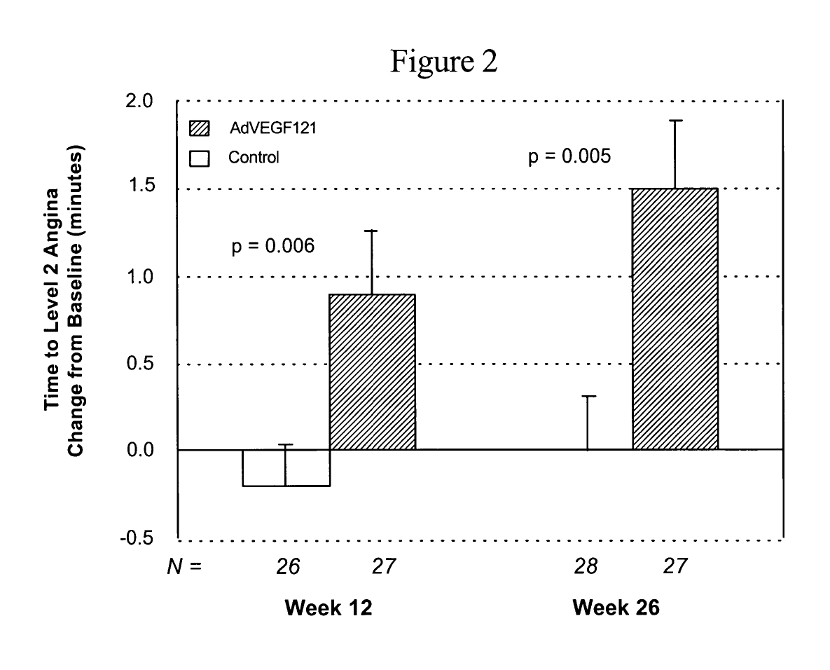

Patient screening, enrollment, treatment, and response, as well as statistical testing, were conducted as described in Example 1. All reported p-values compare results between treatment groups. Group A included 27 patients and Group B included 29 patients, although evaluable data was not available for all patients at all time points. A parameter used to assess the efficacy of the treatment was the change in time to onset of Level 2 angina or termination of ETT in the absence of Level 2 angina at 12 weeks and 26 weeks post-treatment, as compared to time to onset of Level 2 angina before treatment (i.e., baseline). The mean time to onset of Level 2 angina before treatment was 4.1 minutes in Group A and 4.2 minutes in Group B. The results of this efficacy measurement are set forth in FIG. 2. At 12 weeks post-treatment, approximately a 22% increase in time to Level 2 angina was observed in Group A, as compared to the time to onset of Level 2 angina before treatment, a significant change compared to control Group B (p=0.006). At 26 weeks post-treatment, approximately a 37% increase in time to onset of Level 2 angina was observed in Group A, as compared to the time to onset of Level 2 angina before treatment, a significant change compared to control Group B (p=0.003).

### EXAMPLE 3

This example demonstrates the safety and efficacy of a pharmaceutical composition of the invention delivered through minimally invasive surgery versus maximum medical treatment in the treatment of patients suffering from advanced coronary artery disease, as measured by comparing time of total exercise duration during the exercise tolerance test (ETT) before and after treatment in accordance with the invention.

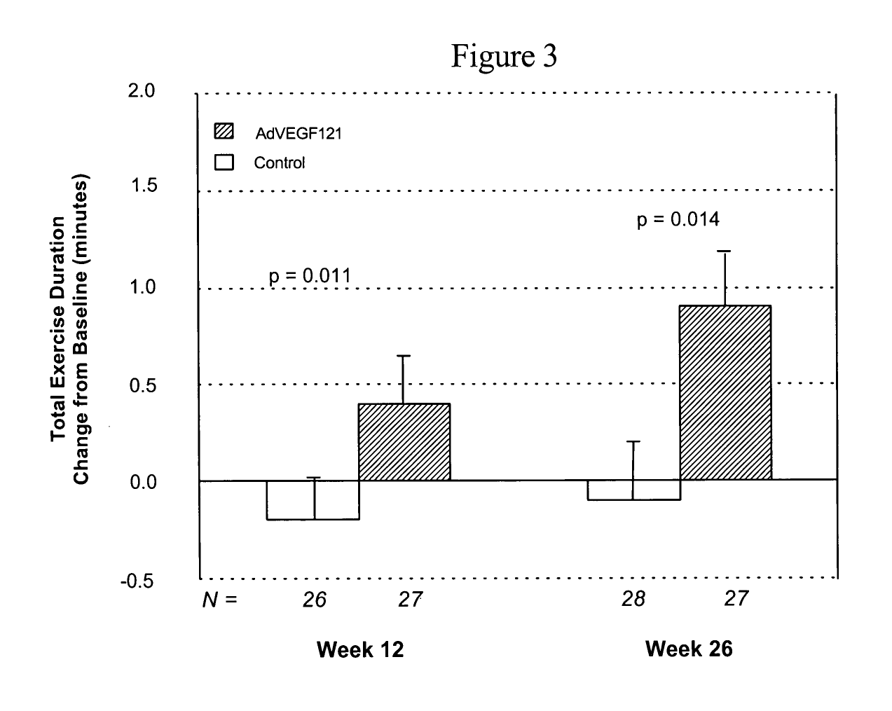

Patient screening, enrollment, treatment, and response, as well as statistical testing, were conducted as described in Example 1. All reported p-values compare results between treatment groups. Another parameter used to assess the efficacy of the treatment was the change in time of total exercise duration during ETT at 12 weeks and 26 weeks post-treatment, as compared to time of total exercise duration during ETT before treatment (i.e., baseline). The mean time of total exercise duration during ETT before treatment was 5.6 minutes for Group A and 5.4 minutes for Group B. The results of this efficacy measurement are set forth in FIG. 3. At 12 weeks post-treatment, approximately a 7% increase in time of total exercise duration was observed in Group A, as compared to baseline, a significant change compared to control Group B (p=0.008). At 26 weeks post-treatment, approximately a 16% increase in time of total exercise duration was observed in Group A, as compared baseline, a significant change compared to control Group B (p=0.015).

### EXAMPLE 4

This example demonstrates the safety and efficacy of a pharmaceutical composition of the invention delivered through minimally invasive surgery in accordance with the treatment method of the invention versus maximum medical treatment in the treatment of patients suffering from advanced coronary artery disease, as measured by an improvement in angina class as assigned in accordance with the Canadian Cardiovascular Society (CCS) angina classification.

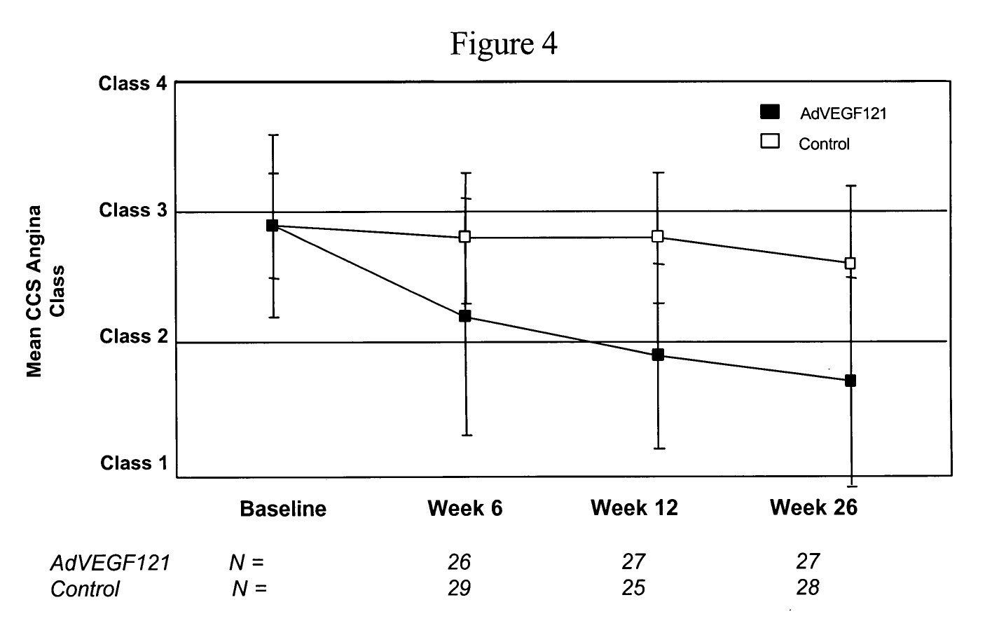

Patient screening, enrollment, treatment, and response, as well as statistical testing, were conducted as described in Example 1. All reported p-values compare results between treatment groups. The CCS angina class was established for Group A and Group B subjects before treatment (i.e., baseline). The CCS angina class was dichotomized into those who improved at least one angina class (i.e., a decrease of at least one class) from baseline (responders) and those with deterioration in angina class, no change in angina class, or no CCS data (nonresponders). The responder/nonresponder rates were summarized by treatment group at Weeks 6, 12, and 26 post-treatment. At Week 6, the responder rate for Group A was approximately 48%, while the responder rate for Group B was approximately 10% (p=0.003). At Week 12, Group A demonstrated approximately an 82% responder rate, as compared to about a 14% responder rate in Group B (p<0.001). At Week 26, the responder rate for Group A was approximately 85%, while the responder rate for Group B was approximately 24% (p<0.001). The mean CCS angina class for each treatment group was calculated at Weeks 6, 12, and 26 post-treatment, and is plotted in FIG. 4.

### EXAMPLE 5

This example demonstrates the safety and efficacy of a pharmaceutical composition of the invention delivered through minimally invasive surgery in accordance with the treatment method of the invention versus maximum medical treatment in the treatment of patients suffering from advanced coronary artery disease, as measured by an improvement in time to onset of angina during the exercise tolerance test (ETT).

Patient screening, enrollment, treatment, and response, as well as statistical testing, were conducted as described in Example 1. The time to onset of angina during ETT was established for Group A and Group B subjects before treatment (i.e., baseline) and after treatment. The magnitude of improvement in time to angina was measured as either at least a 1 minute increase or at least a 3 minute increase in time to angina during ETT (i.e., responders). The responder rates were summarized by treatment group at Weeks 12 and 26 post-treatment. At Week 12, the time to onset of angina improved by at least 1 minute for approximately 48% of the patients in Group A and approximately 14% of the patients in Group B. At the same time point, the time to onset of angina improved by at least 3 minutes for approximately 19% of the patients in Group A and approximately 4% of the patients in Group B. At Week 26, the time to onset of angina improved by at least 1 minute for approximately 52% of the patients in Group A and approximately 29% of the patients in Group B. At the same time point, the time to onset of angina improved by at least 3 minutes in approximately 30% of the patients in Group A and approximately 7% of the patients in Group B.

### EXAMPLE 6

This example demonstrates the safety and efficacy of a pharmaceutical composition of the invention delivered through minimally invasive surgery in accordance with the treatment method of the invention versus maximum medical treatment in the treatment of patients suffering from advanced coronary artery disease, as measured by an improvement in scores reported in the Seattle Angina Questionnaire (SAQ).

Patient screening, enrollment, treatment, and response, as well as statistical testing, were conducted as described in Example 1. To evaluate certain quality-of-life parameters following treatment, the SAQ was administered to subjects in both treatment groups. In particular, the mean change in scores from baseline was measured at Weeks 6, 12, and 26 for three SAQ scales: angina stability, angina frequency, and disease perception. The angina stability score increased by approximately 74% from baseline in Group A subjects at 6 weeks post-treatment. At Week 12, the angina stability score increased approximately 87% from baseline in Group A. At Week 26, the angina stability score increased by approximately 76% from baseline in Group A. At Week 6, an 87% increase from baseline in the angina frequency score was reported for Group A. At Week 12, approximately a 72% increase from baseline in the angina frequency score was reported for Group A, and approximately a 73% increase from baseline in the same score was reported at Week 26. The disease perception score increased by about 45% from baseline in Group A at Week 6, and by about 72% from baseline at Week 12. At Week 26, the disease perception score increased by about 80% from baseline in Group A. In all instances, the change from baseline for Group A was a significant change compared to control Group B (p≦0.001).

### EXAMPLE 7

This example illustrates the tolerability and feasibility of administering a pharmaceutical composition of the invention via a navigational injection device to treat a patient suffering from advanced coronary artery disease in accordance with the treatment method of the invention.

Patients were screened to allow initial assessment of those who may qualified for randomization into the study (i.e., those with advanced coronary disease). Baseline values were then established for each qualified patient with respect to ETT, CCS, and SAQ.

Ten patients with advanced CAD and severe angina pectoris (CCS angina class III-IV, excluding prolonged unstable angina at rest) were enrolled after the screening and baseline procedures at approximately 3 to 4 study centers in different geographic areas in North America. These patients had been treated with maximum medical therapy for at least two months prior to the present study, but the disease failed to be completely controlled, resulting in unacceptable lifestyle limitations. Maximum medical therapy included the following medications (unless hemodynamic parameters or intolerance contraindicated their use): nitroglycerine, platelet aggregation inhibitors (e.g., aspirin, ticlopidine, or clopidogrel), and medications from two of the following: long-acting nitrates, calcium-channel blockers, and beta-blockers. In addition to the above, patients were not candidates for coronary artery bypass grafting, percutaneous transluminal coronary angioplasty, or intravascular stenting. Also, other revascularization procedures and/or heart transplantation were not planned in the 12 weeks following randomization. These patients were then randomized in a 1:2 (placebo:treatment) fashion. Placebo (15 injections, each 100 μl of diluent) (Group A) or a single dose of the pharmaceutical composition (15 endocardial injections, each 100 μl of pharmaceutical composition, totaling 4×10¹⁰ pu adenoviral vector) (Group B) was administered evenly (injections spaced approximately 1.5 to 2 cm apart, not more than 2 cm between injections) across the left ventricle of the cardiac muscle via catheter injection (Biosense® intramyocardial injection device available from Biosense, Inc.) in a double-blind fashion. Patients were hospitalized for a minimum of 24 hours. Vital signs and electrocardiographic signs of ischemia were monitored daily, and managed according to the current standard of care in use at the study site, until recovery from catheterization.

Assessment of vector levels in the plasma, before and within 15 minutes after the completion of the injection procedure, was performed at selected sites. The assessment of plasma VEGF levels also were performed at baseline, day 1, day 3, week 1, week 4, and week 24 post-treatment at selected sites. Adenoviral cultures were performed at 24 hours post-procedure at selected sites. Follow-up assessments were performed at Week 12 post-administration, although assessments were performed at any time for safety reasons. The study was completed for data analysis when all patients withdrew or completed procedures up to and including the Week 12 visit. All patients were followed up at 1 year (Week 52) for general safety assessment (physical examination, noninvasive cancer screen, and body weight) and incidence of cardiac events (hospitalization for MI or unstable angina).

The primary parameters to test efficacy was a survey to assess the feasibility of administering the pharmaceutical composition via catheter injection (specifically via the Biosense® intramyocardial injection device, available from Biosense, Inc.) completed by the interventional cardiologist. The safety and tolerability of the pharmaceutical composition also was assessed. Feasibility was determined based on a Case Report Form completed by the interventional cardiologist on the day of drug administration. The following parameters were evaluated: elapsed time between start of catheterization (time of skin incision) and end of catheterization (initiation of skin closure or external compression procedures after removal of sheath) (mean 5.7 hours, median 3.3 hours), elapsed time between start of catheterization (start of procedure) and removal of last injection catheter (mean 2.4 hours, median 2.5 hours), elapsed time between insertion of first injection catheter into sheath and removal of last injection catheter from sheath (mean 51.4 minutes, median 44 minutes), elapsed time between first and last injection (mean 38.3 minutes, median 33.5 minutes), and total fluoroscopy time (mean 29.9 minutes, median 17.1 minutes).

The safety and tolerability of intramyocardial administration of the pharmaceutical composition was assessed. Five patients developed complications potentially related to the procedure, all of which were resolved within a few days. The complications included drowsiness, dark stool, peripheral edema, scattered rhonchi, pleural effusions, ventricular tachycardia, premature ventricular contractions (PVC), back pain, groin eccymosis, vomiting, bradycardia, cough, and UR1. One patient developed an adverse event (fever), which was considered as related to treatment. All adenoviral cultures from urine and throat swabs were negative.

Secondary parameters were evaluated at Week 12 and included the change from baseline in the following: ETT parameters (i.e., time to onset of at least 1 mm additional ST-segment depression, or termination of ETT in the absence of at least 1 mm additional ST-segment depression; total exercise duration; time to onset of Level 2 angina, or termination of ETT in the absence of Level 2 angina; peak rate pressure product (heart rate×systolic pressure); maximum depth of ST-segment depression; rate pressure product at onset of Level 2 angina, or peak rate pressure product in the absence of Level 2 angina; and rate pressure product at onset of at least 1 mm additional ST-segment depression, or peak rate pressure product in the absence of at least 1 mm additional ST-segment depression); CCS angina class; and the SAQ.

The Case Report From completed by the interventional cardiologist on the day of administration of the pharmaceutical composition provided primarily narrative information. For questions of a quantitative nature, summary statistics were provided to aid in the subjective determination of feasibility. Feasibility was assessed based on blinded data. At the end of the study, feasibility data was summarized by treatment group. Secondary parameters were summarized within each treatment group. Additionally, 95% confidence intervals were provided for the change from baseline measures. The CCS angina class was dichotomized into those who improved angina class from baseline (responders) and those with deterioration in angina class, no change in angina class, or no CCS data (nonresponders). The responder/nonresponder rates were summarized by treatment group at Week 12.

The results demonstrated that the inventive method and pharmaceutical compositions were well tolerated by the human subjects. Due to the small sample size, statistical analysis of secondary efficacy results was not performed.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Of course, variations of those preferred embodiments will become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating coronary artery disease in a human patient comprising directly injecting into an ischemic cardiac muscle, via multiple injections to different points of the cardiac muscle using a catheter comprising a navigational system, a dose of a pharmaceutical composition comprising (a) a pharmaceutically acceptable carrier and (b) a replication-deficient adenoviral vector comprising a nucleic acid sequence encoding an angiogenic peptide operably linked to a promoter, wherein the dose comprises about 1×10⁸ to about 4×10¹¹ particle units of the replication-deficient adenoviral vector, whereby the coronary artery disease is treated, as evidenced by one or more of the following:
(i) at least a 5% increase in time to onset of at least 1 mm additional ST-segment depression on exercise electrocardiograms (ECG) or termination of exercise tolerance test (ETT) in the absence of at least 1 mm additional ST-segment depression at 12 weeks post-treatment compared to time to onset of at least 1 mm additional ST-segment depression on ECG before treatment,
(ii) at least a 10% increase in time to onset of Level 2 angina or termination of ETT in the absence of Level 2 angina at 12 weeks post-treatment compared to time of onset before treatment,
(iii) at least a 5% increase in time of total exercise duration during ETT at 12 weeks post-treatment compared to time of total exercise duration during ETT before treatment,
(iv) a decrease of at least one angina class as assigned by the Canadian Cardiovascular Society Angina Classification at 12 weeks post-treatment compared to the angina class before treatment,
(v) at least a 50% increase in the Seattle Angina Questionnaire angina stability score or angina frequency score, reported by the human patient at 6 weeks post-treatment compared to the angina stability score and angina frequency score reported by the human patient before treatment, or
(vi) at least a 30% increase in the Seattle Angina Questionnaire disease perception score reported by the human patient at 6 weeks post-treatment compared to the disease perception score reported by the human patient before treatment.

2. A method of treating coronary artery disease in a human patient comprising directly injecting into an ischemic cardiac muscle, via multiple injections to different points of the cardiac muscle using a catheter comprising a navigational system, a dose of a pharmaceutical composition comprising (a) a pharmaceutically acceptable carrier and (b) a replication-deficient adenoviral vector comprising a nucleic acid sequence encoding an angiogenic peptide operably linked to a promoter, wherein the dose comprises the replication-deficient adenoviral vector in a quantity that achieves a concentration of the angiogenic peptide of at least about 100 pg angiogenic peptide per 1 mg of total protein at the injection site, whereby the coronary artery disease is treated as evidenced by one or more of the following:
(i) at least a 5% increase in time to onset of at least 1 mm additional ST-segment depression on exercise electrocardiograms (ECG) or termination of ETT in the absence of at least 1 mm additional ST-segment depression at 12 weeks post-treatment compared to time to onset of at least 1 mm additional ST-segment depression on ECG before treatment,
(ii) at least a 10% increase in time to onset of Level 2 angina or termination of ETT in the absence of Level 2 angina at 12 weeks post-treatment compared to time of onset before treatment,
(iii) at least a 5% increase in time of total exercise duration during ETT at 12 weeks post-treatment compared to time of total exercise duration during ETT before treatment,
(iv) a decrease of at least one angina class as assigned by the Canadian Cardiovascular Society Angina Classification at 12 weeks post-treatment compared to the angina class before treatment,
(v) at least a 50% increase in the Seattle Angina Questionnaire angina stability score or angina frequency score, reported by the human patient at 6 weeks post-treatment compared to the angina stability score and angina frequency score reported by the human patient before treatment, or
(vi) at least a 30% increase in the Seattle Angina Questionnaire disease perception score reported by the human patient at 6 weeks post-treatment compared to the disease perception score reported by the human patient before treatment.

3. The method of claim 1, wherein the angiogenic peptide is a vascular endothelial growth factor (VEGF).

4. The method of claim 3, wherein the angiogenic peptide is selected from the group consisting of VEGF₁₄₅ and VEGF₁₈₉.

5. The method of claim 3, wherein the angiogenic peptide is VEGF₁₂₁.

6. The method of claim 3, wherein the angiogenic peptide is VEGF₁₆₅.

7. The method of claim 2, wherein the concentration of the angiogenic peptide is about 100 pg to about 1400 pg angiogenic peptide per 1 mg of total protein at the injection site.

8. The method of claim 2, wherein the concentration of the angiogenic peptide is about 2 ng to about 6 ng angiogenic peptide per 1 mg of total protein at the injection site.

9. The method of claim 2, wherein the concentration of the angiogenic peptide 1 cm from the site of injection is about 5 pg to about 150 pg angiogenic peptide per 1 mg of total protein at the injection site.

10. The method of claim 1, wherein the multiple injections comprise about 5 to about 50 injections.

11. The method of claim 1, wherein the volume of each injection is about 50 μl to about 500 μl.

12. The method of claim 1, wherein the volume of the dose of the pharmaceutical composition is about 1 to about 5 ml.

13. The method of claim 1, wherein the multiple injections are spaced about 1 to about 2.5 cm apart.

14. The method of claim 1, wherein the dose comprises about 1×10¹⁰ to about 9×10¹⁰ particle units of the replication-deficient adenoviral vector.

15. The method of claim 1, wherein each injection comprises about 1×10⁹ to about 5×10⁹ particle units of the replication-deficient adenoviral vector.

16. The method of claim 1, wherein the volume of each injection is about 50 to about 150 μl.

17. The method of claim 1, wherein each injection comprises about 1×10⁷ Pu to about 5×10⁷ particle units of the replication-deficient adenoviral vector per 1 μl of the pharmaceutical composition.

18. The method of claim 1, whereby the treatment of the coronary artery disease is evidenced by at least a 20% increase in time to onset of at least 1 mm additional ST-segment depression on exercise electrocardiograms (ECG) or termination of ETT in the absence of at least 1 mm additional ST-segment depression at 26 weeks post-treatment compared to time to onset of at least 1 mm additional ST-segment depression on ECG before treatment.

19. The method of claim 1, whereby the treatment of the coronary artery disease is evidenced by at least a 20% increase in time to onset of Level 2 angina or termination of ETT in the absence of Level 2 angina at 26 weeks post-treatment compared to time of onset before treatment.

20. The method of claim 1, whereby the treatment of the coronary artery disease is evidenced by at least a 10% increase in time of total exercise duration during ETT at 26 weeks post-treatment compared to time of total exercise duration during ETT before treatment.

21. The method of claim 1, whereby the treatment of the coronary artery disease is evidenced by a decrease of at least two angina classes as assigned by the Canadian Cardiovascular Society Angina Classification at 26 weeks post-treatment compared to the angina class before treatment.

22. The method of claim 1, whereby the treatment of the coronary artery disease is evidenced by at least a 50% increase in the Seattle Angina Questionnaire angina stability score reported by the human patient at 12 or 26 weeks post-treatment compared to the angina stability score reported by the human patient before treatment.

23. The method of claim 1, whereby the treatment of the coronary artery disease is evidenced by at least a 50% increase in the Seattle Angina Questionnaire angina frequency score reported by the human patient at 12 or 26 weeks post-treatment compared to the angina frequency score reported by the human patient before treatment.

24. The method of claim 1, whereby the treatment of the coronary artery disease is evidenced by at least a 50% increase in the Seattle Angina Questionnaire disease perception score reported by the human patient 12 or 26 weeks post-treatment compared to the disease perception score reported by the human patient before treatment.
